(19) United States
(12) Patent Application Publication
(10) Pub. No.: US 2025/0050241 A1
(43) Pub. Date: Feb. 13, 2025

(54) CHROMATOGRAPHIC SEPARATION PROCESS FOR EFFICIENT PURIFICATION OF POLYUNSATURATED FATTY ACIDS

(71) Applicant: BASF SE, Ludwigshafen am Rhein (DE)

(72) Inventors: Pedro SÁ GOMES, Ludwigshafen am Rhein (DE); Angus MORRISON, Breasclete (GB); Johan Fredrik BILLING, Ludwigshafen am Rhein (DE); Iseabal MACARTHUR, Breasclete (GB)

(21) Appl. No.: 18/718,485

(22) PCT Filed: Dec. 16, 2022
(86) PCT No.: PCT/EP2022/086458

§ 371 Date: Jun. 11, 2024

(30) Foreign Application Priority Data
Dec. 17, 2021 (EP) 21215675.6

(51) Int. Cl. B01D 15/18; B01D 15/20; B01J 20/28; B01J 20/287

(52) U.S. Cl. CPC B01D 15/1821; B01D 15/20; B01J 20/28004; B01J 20/28011; B01J 20/28016; B01J 20/28057; B01J 20/287

(57) ABSTRACT

The present invention relates to an improved chromatographic separation process for purification of a polyunsaturated fatty acid (PUFA) product and derivatives thereof. In particular, the present invention relates to a particularly efficient chromatographic separation process that employs silica having particular physical characteristics as an adsorbent phase for purifying a PUFA or derivative thereof from a feed mixture.

### FIELD OF THE INVENTION

The present invention relates to an improved chromatographic separation process for purification of a polyunsaturated fatty acid (PUFA) product and derivatives thereof. In particular, the present invention relates to a particularly efficient chromatographic separation process that employs silica having particular physical characteristics as an adsorbent phase for purifying a PUFA or derivative thereof from a feed mixture.

### BACKGROUND INFORMATION

Fatty acids, in particular PUFAs, and their derivatives are precursors for biologically important molecules, which play an important role in the regulation of biological functions such as platelet aggregation, inflammation and immunological responses. Thus, PUFAs and their derivatives may be therapeutically useful in treating a wide range of pathological conditions including CNS conditions; neuropathies, including diabetic neuropathy; cardiovascular diseases; general immune system and inflammatory conditions, including inflammatory skin diseases.

PUFAs are found in natural raw materials, such as vegetable oils and marine oils. Such PUFAs are, however, frequently present in such oils in admixture with saturated fatty acids and numerous other impurities. PUFAs should therefore desirably be purified before nutritional or pharmaceutical uses.

Unfortunately, PUFAs are extremely fragile. Thus, when heated in the presence of oxygen, they are prone to isomerization, peroxidation and oligomerization. The fractionation and purification of PUFA products to prepare pure fatty acids is therefore difficult. Distillation, even under vacuum, can lead to non-acceptable product degradation.

Chromatographic separation techniques are well known to those of skill in the art. Chromatographic separation techniques involving stationary bed systems and simulated or actual moving bed systems are both familiar to one of skill in the art.

In a conventional stationary bed chromatographic system, a mixture whose components are to be separated percolates through a container. The container is generally cylindrical, and is typically referred to as the column. The column contains a packing of a porous, adsorbent material (generally called the stationary phase) exhibiting a high permeability to fluids. The percolation velocity of each component of the mixture depends on the physical properties of that component so that the components exit from the column successively and selectively. Thus, some of the components tend to fix strongly to the stationary phase and thus will percolate slowly, whereas others tend to fix weakly and exit from the column more quickly. Many different stationary bed chromatographic systems have been proposed and are used for both analytical and industrial production purposes.

Simulated and actual moving bed chromatography are known techniques, familiar to those of skill in the art. The principle of operation involves countercurrent movement of a liquid eluent (or desorbent) phase and a solid adsorbent phase. This operation allows minimal usage of solvent and specific adsorbent inventory making the process economically viable. Such separation technology has found several applications in diverse areas, including hydrocarbons, industrial chemicals, oils, sugars and APIs.

Thus, a simulated moving bed chromatography apparatus consists of a number of individual columns containing adsorbent which are connected together in series. Eluent is passed through the columns in a first direction. The injection points of the feedstock and the eluent, and the separated component collection points in the system, are periodically shifted by means of a series of valves or a single multi-position valve. The overall effect is to simulate the operation of a single column containing a moving bed of the solid adsorbent, the solid adsorbent moving in a countercurrent direction to the flow of eluent. Thus, a simulated moving bed system consists of columns which, as in a conventional stationary bed system, contain stationary beds of solid adsorbent through which eluent is passed, but in a simulated moving bed system the operation is such as to simulate a continuous countercurrent moving bed.

A typical simulated moving bed chromatography apparatus is illustrated with reference to FIG. 1. The concept of a simulated or actual moving bed chromatographic separation process is explained by considering a vertical chromatographic column containing stationary phase S divided into sections, more precisely into four superimposed sub-zones I, II, III and IV going from the bottom to the top of the column. The eluent is introduced at the bottom at IE by means of a pump P. The mixture of the components A and B which are to be separated is introduced at IA+B between sub-zone II and sub-zone III. An extract containing mainly B is collected at SB between sub-zone I and sub-zone II, and a raffinate containing mainly A is collected at SA between sub-zone III and sub-zone IV.

In the case of a simulated moving bed system, a simulated downward movement of the stationary phase S is caused by movement of the introduction and collection points relative to the solid phase. In the case of an actual moving bed system, simulated downward movement of the stationary phase S is caused by movement of the various chromatographic columns relative to the introduction and collection points. In FIG. 1, eluent flows upward and mixture A+B is injected between sub-zone II and sub-zone III. The components will move according to their chromatographic interactions with the stationary phase, for example adsorption on a porous medium. The component B that exhibits stronger affinity to the stationary phase (the slower running component) will be more slowly entrained by the eluent and will follow it with delay. The component A that exhibits the weaker affinity to the stationary phase (the faster running component) will be easily entrained by the eluent. If the right set of parameters, especially the flow rate in each sub-zone, are correctly estimated and controlled, the component A exhibiting the weaker affinity to the stationary phase will be collected between sub-zone III and sub-zone IV as a raffinate and the component B exhibiting the stronger affinity to the stationary phase will be collected between sub-zone I and sub-zone II as an extract.

It will therefore be appreciated that the conventional simulated moving bed system schematically illustrated in FIG. 1 is limited to binary fractionation.

The benefits of other related, so-called simulated moving bed non-conventional operating modes, such as Improved-SMB, Sequential-SMB, Varicol, Powerfeed, Modicon, MCSGP, Outlet Swing Stream-OSS, JO or pseudo SMB, among others, is known and can be derived from the conventional simulated moving bed process by a skilled expert, as detailed elsewhere (see e.g. Sa Gomes and Rodrigues, Chemical Engineering and Technology Special Issue: Preparative Chromatography and Downstream Processing, 2012, 35(1), 17-34, the contents of which are incorporated by reference herein in their entirety). Processes and equipment for simulated moving bed chromatography are described in several patents, including U.S. Pat. Nos. 2,985,589, 3,696,107, 3,706,812, 3,761,533, FR-A-2103302, FR-A-2651148 and FR-A-2651149, the entirety of which are incorporated herein by reference. The topic is also dealt with at length in “Preparative and Production Scale Chromatography”, edited by Ganetsos and Barker, Marcel Dekker Inc, New York, 1993, the entirety of which is incorporated herein by reference.

An actual moving bed system is similar in operation to a simulated moving bed system. However, rather than shifting the injection points of the feed mixture and the eluent, and the separated component collection points by means of a system of valves or a single multiposition valve, instead a series of adsorption units (i.e. columns) are physically moved relative to the feed and drawoff points. Again, operation is such as to simulate a continuous countercurrent moving bed.

Processes and equipment for actual moving bed chromatography are described in several patents, including U.S. Pat. Nos. 6,979,402, 5,069,883 and 4,764,276, the entirety of which are incorporated herein by reference.

Purification of PUFA products is particularly challenging. Thus, many suitable feedstocks for preparing PUFA products are extremely complex mixtures containing a large number of different components with very similar retention times in chromatography apparatuses. It is therefore very difficult to separate certain PUFAs from such feedstocks. However, a high degree of purity of PUFA products is required, particularly for pharmaceutical and nutraceutical applications. Historically, therefore, distillation has been used when high purity PUFA products are required. There are, however, significant drawbacks to using distillation as a separation technique for delicate PUFAs as discussed above.

Published international patent application WO 2011/080503, the entirety of which is incorporated herein by reference, discloses an SMB separation process for recovering a PUFA product from a feed mixture efficiently and in very high purity. Adaptations of this particular set-up are described in WO 2013/005046, WO 2013/005047, WO 2013/005048, WO 2013/005051, WO 2013/005052 and WO 2014/108686, all of which are incorporated herein by reference. It has been found, however, that large volumes of eluent are often required in such chromatographic systems when operated at low pressure (i.e., less than 20 bar, and preferably less than 10 bar). This is in contrast to high performance liquid chromatography (HPLC) systems which typically operate at higher pressures (i.e., 20-100 bar) and which can extract PUFA products from feed mixtures with both a high productivity and a low dilution (i.e. amount) of solvent required. Operating at high pressures however has several disadvantages in large-scale commercial processes. In particular, the stationary phase (e.g. silica) in the chromatographic columns is put under increased stress and must be replaced on a more frequent basis.

Accordingly, there is a need to provide a more efficient chromatographic separation process for purifying PUFA products from feed mixtures at a low operating pressure, such that less eluent is required to extract the PUFA products.

### SUMMARY OF THE INVENTION

The present inventors have surprisingly found that employing a silica adsorbent phase having particular physical characteristics can result in enhanced resolution of the peaks representing different PUFA products in a typical PUFA-containing feedstock, whilst operating at low pressure. This enhanced resolution between the peaks results in a more efficient separation, requiring a lower dilution of eluent. It has even been found that a lower dilution of eluent can be achieved than when employing HPLC processes, whilst also achieving an acceptable (and comparatively, surprisingly good) level of productivity.

The present invention therefore provides a chromatographic separation process for recovering a polyunsaturated fatty acid (PUFA) product from a feed mixture, which comprises introducing the feed mixture into a chromatography apparatus comprising one or more chromatographic columns containing:
(a) a liquid eluent phase which is an aqueous organic solvent; and
(b) a solid adsorbent phase which is a C18-bonded silica,
wherein the pressure within the one or more chromatographic columns is less than 20 bar, thereby resulting in purification of the feed mixture, further wherein:
(1) the silica has an average particle diameter of from 230 to 270 μm and a Dv(10) of 160 μm or greater; and/or
(2) the silica has a carbon loading of from 15 to 24 wt %; and/or
(3) the silica has a surface area of 500 m²/g or less.

In a particular embodiment, the present invention provides a chromatographic separation process for recovering a polyunsaturated fatty acid (PUFA) product from a feed mixture, which comprises introducing the feed mixture into a chromatography apparatus comprising one or more chromatographic columns containing:
(a) a liquid eluent phase which is an aqueous organic solvent; and
(b) a solid adsorbent phase which is a C18-bonded silica with a carbon loading of from 15 to 24 wt %,
wherein the pressure within the one or more chromatographic columns is less than 20 bar, thereby resulting in purification of the feed mixture, and further wherein:
(1) the silica has an average particle diameter of from 230 to 270 μm and a Dv(10) of 160 μm or greater; and/or
(2) the silica has a surface area of 500 m²/g or less.

### BRIEF DESCRIPTION OF THE FIGURES

FIG. 1 illustrates the basic principles of a simulated or actual moving bed process for separating a binary mixture.

FIG. 2 illustrates a chromatographic separation step, which comprises two simulated or actual moving bed processes, to separate EPA from faster and slower running impurities (i.e. more polar and less polar impurities).

FIG. 3 illustrates a chromatographic separation step, which comprises two simulated or actual moving bed processes, to separate DHA from faster and slower running impurities (i.e. more polar and less polar impurities).

FIG. 4 illustrates a chromatographic separation step, which comprises two simulated or actual moving bed processes, to separate EPA from faster and slower running impurities (i.e. more polar and less polar impurities).

FIG. 5 illustrates a chromatographic separation step, which comprises two simulated or actual moving bed processes, to separate DHA from faster and slower running impurities (i.e. more polar and less polar impurities).

FIG. 6 illustrates a chromatographic separation step, which comprises two simulated or actual moving bed processes, to separate EPA from faster and slower running impurities (i.e. more polar and less polar impurities).

FIG. 7 illustrates a chromatographic separation step, which comprises two simulated or actual moving bed processes, to separate DHA from faster and slower running impurities (i.e. more polar and less polar impurities).

FIG. 8 illustrates a chromatographic separation step, which comprises two simulated or actual moving bed processes, to separate EPA from faster and slower running impurities (i.e. more polar and less polar impurities).

FIG. 9 illustrates a chromatographic separation step, which comprises two simulated or actual moving bed processes, to separate EPA from faster and slower running impurities (i.e. more polar and less polar impurities).

FIG. 10 illustrates three ways in which a chromatographic separation step which comprises two simulated or actual moving bed processes may be carried out.

FIG. 11 illustrates a chromatographic separation step to separate EPA from faster and slower running impurities (i.e. more polar and less polar impurities).

FIG. 12 illustrates the total contribution to particle volume of a silica sample as a function of particle diameter for different C18 silica types. (A) Silica 1 (reference silica), two separate batches, and silica 2. (B) Silica 1 (reference silica), two separate batches, and silicas 3 and 4.

FIG. 13 illustrates scanning electron microscope images of: (A) silica 1; (B) silica 2; and (C) silica 4.

FIG. 14 illustrates pulse tests to show the asymmetry in the EPA peak during purification of an EPA-containing feedstock in a chromatography column using different silicas.

### DETAILED DESCRIPTION

In its most general sense, the present invention provides a chromatographic separation process for recovering a polyunsaturated fatty acid (PUFA) product from a feed mixture, which comprises introducing the feed mixture into a chromatography apparatus comprising one or more chromatographic columns containing:
(a) a liquid eluent phase which is an aqueous organic solvent; and
(b) a solid adsorbent phase which is a C18-bonded silica,
wherein the pressure within the one or more chromatographic columns is less than 20 bar, thereby resulting in purification of the feed mixture.

### The Solid Adsorbent Phase

The solid adsorbent phase is typically a reverse-phase silica. The solid adsorbent phase is typically a C18-bonded silica gel. Preferably, the solid adsorbent phase is a reverse phase C18-bonded silica gel. The adsorbent phase is typically non-polar.

The chromatography apparatus comprises one or more chromatographic columns, for example 1, 2, 3, 4, 5, 6, 7, 8, 9, or 10 columns. In some embodiments the number of columns is typically one. In other embodiments the number of columns is typically more than one, preferably 4 or more, more preferably 6 or more, even more preferably 8 or more, for example 4, 5, 6, 7, 8, 9, or 10 columns. Typically, there are no more than 25 columns, preferably no more than 20, more preferably no more than 15. When more than one chromatographic column is used, each chromatographic column may contain the same or a different adsorbent. Typically, when more than one chromatographic column is used each column contains the same adsorbent.

The shape of the solid adsorbent phase material may be, for example, spherical or non-spherical beads, preferably substantially spherical beads.

The C18-bonded silica has particular physical characteristics.

In a first embodiment of the present invention, the C18-bonded silica has an average particle diameter of from 230 to 270 μm and a Dv(10) of 160 μm or greater.

In a second embodiment of the present invention, the C18-bonded silica has a carbon loading of from 15 to 24 wt %.

In a third embodiment of the present invention, the C18-bonded silica has a surface area of 500 m²/g or less.

Each of these particular physical characteristics of the silica has been found to lead to increased resolution between different peaks in the separation of a PUFA-containing feedstock, where each peak corresponds to a different PUFA product. This in turn allows for a more efficient purification of a desired PUFA product in high yield whilst using less solvent. This reduced use of solvent is both more cost-effective for a separation that is carried out on an industrial scale, and more environmentally friendly. Moreover, the ability to achieve these advantages whilst employing relatively large particles of silica enables a lower pressure of eluent to be used in the system. This has additional advantages in terms of cost savings and lifetime of the apparatus, and the reduced frequency with which the solid adsorbent phase must be replaced.

Thus, in a first embodiment, the present invention provides a chromatographic separation process for recovering a polyunsaturated fatty acid (PUFA) product from a feed mixture, which comprises introducing the feed mixture into a chromatography apparatus comprising one or more chromatographic columns containing:
(a) a liquid eluent phase which is an aqueous organic solvent; and
(b) a solid adsorbent phase which is a C18-bonded silica having an average particle diameter of from 230 to 270 μm and a Dv(10) of 160 μm or greater,
wherein the pressure within the one or more chromatographic columns is less than 20 bar, thereby resulting in purification of the feed mixture.

As defined herein, the “average particle diameter” refers to the volume moment mean of the particles (also referred to as D[4,3], the volume-weighted mean diameter or the De Brouckere mean diameter). Dv(10) refers to the 10^{th} percentile of particle diameter in a plot of cumulative volume distribution of the silica particles against increasing particle diameter. The volume moment mean is particularly sensitive to the number of coarse particles (i.e. particles of large size) present in a sample, and the Dv(10) value is particularly sensitive to the number of fine particles (i.e. particles of small size) present in a sample. Thus, taken together, the volume moment mean and the Dv(10) value represent an effective characterisation of both the average particle size and the particle size distribution of the silica.

The volume moment mean and Dv(10) are typically measured by laser diffraction, e.g. using the standard method ISO 13320:2020. Details of laser diffraction are discussed for example at https://www.malvernpanalacal.com/en/products/technolog:/light-scattering/laser-diffraction (accessed 15 Mar. 2021), the contents of which are herein incorporated by reference in their entirety.

It is preferred that the average particle diameter is reasonably large, so that a low pressure can be employed in the chromatographic separation. However, it is also preferred that the particle size distribution is narrow, as this is surprisingly found to enhance the resolution between the different PUFA peaks in the purification of a PUFA-containing feedstock, enabling less eluent to be used in the separation. In particular, it has been surprisingly found that reduction or elimination of a “tail” of particularly fine particles (i.e. particles having a small diameter) from the silica sample results in increased resolution between the PUFA peaks. Thus, it is found that a reasonably high average particle diameter, a high Dv(10) value and a relatively small difference between the average particle diameter and Dv(10) value are all desirable characteristics of the silica for use in this embodiment.

Thus, in the first embodiment, the average particle diameter is preferably from 235 to 265 μm, more preferably from 240 to 260 μm, still more preferably from 245 to 260 μm, and most preferably from 250 to 260 μm.

In the first embodiment, the silica has a Dv(10) which is preferably 165 μm or greater, more preferably 170 μm or greater, even more preferably 175 μm or greater, yet more preferably 180 μm or greater, and most preferably 185 μm or greater. Typically, the silica has a Dv(10) which is 225 μm or less, preferably 220 μm or less, more preferably 215 μm or less, and most preferably 210 μm or less. Thus, typically, the silica has a Dv(10) of from 160 to 225 μm, preferably from 165 to 220 μm, more preferably from 175 to 215 μm, and most preferably from 185 to 210 μm.

Thus, in the first embodiment, the silica preferably has an average particle diameter of from 235 to 265 μm and a Dv(10) of 165 μm or greater, and more preferably has an average particle diameter of from 240 to 260 μm and a Dv(10) of 175 μm or greater, and even more preferably has an average particle diameter of from 245 to 260 μm and a Dv(10) of 180 μm or greater, and most preferably has an average particle diameter of from 250 to 260 μm and a Dv(10) of 185 μm or greater.

Thus, in the first embodiment, the silica preferably has an average particle diameter of from 235 to 265 μm and a Dv(10) of 225 μm or less, and more preferably has an average particle diameter of from 240 to 260 μm and a Dv(10) of 220 μm or less, and even more preferably has an average particle diameter of from 245 to 260 μm and a Dv(10) of 215 μm or less, and most preferably has an average particle diameter of from 250 to 260 μm and a Dv(10) of 210 μm or less.

Thus, in the first embodiment, the silica preferably has an average particle diameter of from 235 to 265 μm and a Dv(10) of from 165 to 225 μm, and more preferably has an average particle diameter of from 240 to 260 μm and a Dv(10) of from 175 to 220 μm, and even more preferably has an average particle diameter of from 245 to 260 μm and a Dv(10) of from 180 to 215 μm, and most preferably has an average particle diameter of from 250 to 260 μm and a Dv(10) of from 185 to 210 μm.

In the first embodiment, the silica typically has a carbon loading (% C) of from 15 to 24 wt %, preferably from 16 to 22 wt %, more preferably from 16.5 to 20 wt %, still more preferably from 17 to 19.5 wt %, and most preferably from 17.5 to 19 wt %, for example from 17.5 to 18 wt %. The carbon loading is a measure of the % of the solid absorbent phase that is carbon.

Typically, substantially all of the carbon content derives from the C18-functionalisation of the silica particles.

The carbon loading of the silica particles is typically measured by combustion analysis, e.g. by using methods of the type described in the standard ISO 21068-2, or variants thereof, or by using the method of Nguyen et al. (Science and Technology Development Journal, 2016, 19(4), 162-166, which is incorporated herein by reference in its entirety).

In the first embodiment, the silica typically has a surface area of less than 500 m²/g, preferably less than 450 m²/g, more preferably less than 400 m²/g, and most preferably less than 350 m²/g. The silica typically has a surface area of greater than 100 m²/g, preferably greater than 150 m²/g, more preferably greater than 200 m²/g, still more preferably greater than 250 m²/g, and most preferably greater than 300 m²/g. Thus, the silica typically has a surface area of from 100 to 500 m²/g, preferably from 200 to 450 m²/g, more preferably from 250 to 400 m²/g, and most preferably from 300 to 350 m²/g.

The surface area of the silica particles is typically measured by BET surface area analysis, e.g. by using the standard method ISO 9277:2010. In the method, the surface area is determined from adsorption data, typically from nitrogen adsorption, using BET theory (Brunauer, Emmett and Teller). The BET method of measuring surface area has been described in detail in “Characterization of Porous Solids and Powders: Surface Area, Pore Size and Density” by Lowell, Shields, Thomas and Thommes, Springer, Dordrecht, 2006 (pub: Springer), which is incorporated herein by reference in its entirety.

The surface area in this context refers to the silica surface and is measured on the bare silica before bonding of the C18 chains.

In a second embodiment, the present invention provides a chromatographic separation process for recovering a polyunsaturated fatty acid (PUFA) product from a feed mixture, which comprises introducing the feed mixture into a chromatography apparatus comprising one or more chromatographic columns containing:
(a) a liquid eluent phase which is an aqueous organic solvent; and
(b) a solid adsorbent phase which is a C18-bonded silica with a carbon loading of from 15 to 24 wt %,
wherein the pressure within the one or more chromatographic columns is less than 20 bar, thereby resulting in purification of the feed mixture.

It has been surprisingly found by the present inventors that silica having a carbon loading in this range provides an improved resolution between the PUFA peaks in the separation of a PUFA product from a feed mixture, irrespective of whether the silica comprises a “tail” of smaller particles (i.e. irrespective of whether the Dv(10) value is lower than as described in the first embodiment above).

In the second embodiment, the carbon loading of the silica is preferably from 16 to 22 wt %, more preferably from 16.5 to 20 wt %, still more preferably from 17 to 19 wt %, and most preferably from 17.5 to 18 wt %, for example about 17.8 wt % or 17.9 wt %. Typically, substantially all of the carbon content derives from the C18-functionalisation of the silica particles.

The carbon loading of the silica particles is typically measured as described above in relation to the first embodiment.

In the second embodiment, typically the average particle diameter is from 230 to 270 μm, preferably from 235 to 265 μm, more preferably from 240 to 260 μm, still more preferably from 245 to 260 μm, and most preferably from 250 to 260 μm.

In the second embodiment, typically the Dv(10) value of the silica is 160 μm or greater, preferably 165 μm or greater, more preferably 170 μm or greater, even more preferably 175 μm or greater, yet more preferably 180 μm or greater, and most preferably 185 μm or greater. Typically, the silica has a Dv(10) which is 225 μm or less, preferably 220 μm or less, more preferably 215 μm or less, and most preferably 210 μm or less. Thus, typically, the silica has a Dv(10) of from 160 to 225 μm, preferably from 165 to 220 μm, more preferably from 175 to 215 μm, and most preferably from 185 to 210 μm.

Thus, in the second embodiment, the silica typically has an average particle diameter of from 230 to 270 μm and a Dv(10) of 160 μm or greater, and preferably has an average particle diameter of from 235 to 265 μm and a Dv(10) of 165 μm or greater, and more preferably has an average particle diameter of from 240 to 260 μm and a Dv(10) of 175 μm or greater, and even more preferably has an average particle diameter of from 245 to 260 μm and a Dv(10) of 180 μm or greater, and most preferably has an average particle diameter of from 250 to 260 μm and a Dv(10) of 185 μm or greater.

Thus, in the second embodiment, the silica typically has an average particle diameter of from 230 to 270 μm and a Dv(10) of 225 μm or less, and preferably has an average particle diameter of from 235 to 265 μm and a Dv(10) of 225 μm or less, and more preferably has an average particle diameter of from 240 to 260 μm and a Dv(10) of 220 μm or less, and even more preferably has an average particle diameter of from 245 to 260 μm and a Dv(10) of 215 μm or less, and most preferably has an average particle diameter of from 250 to 260 μm and a Dv(10) of 210 μm or less.

Thus, in the second embodiment, the silica typically has an average particle diameter of from 230 to 270 μm and a Dv(10) of from 160 to 225 μm, preferably has an average particle diameter of from 235 to 265 μm and a Dv(10) of from 165 to 225 μm, and more preferably has an average particle diameter of from 240 to 260 μm and a Dv(10) of from 175 to 220 μm, and even more preferably has an average particle diameter of from 245 to 260 μm and a Dv(10) of from 180 to 215 μm, and most preferably has an average particle diameter of from 250 to 260 μm and a Dv(10) of from 185 to 210 μm.

The average particle diameter and the Dv(10) value are both typically measured as described above in relation to the first embodiment.

In the second embodiment, the silica typically has a surface area of less than 500 m²/g, preferably less than 450 m²/g, more preferably less than 400 m²/g, and most preferably less than 350 m²/g. The silica typically has a surface area of greater than 100 m²/g, preferably greater than 150 m²/g, more preferably greater than 200 m²/g, still more preferably greater than 250 m²/g, and most preferably greater than 300 m²/g. Thus, the silica typically has a surface area of from 100 to 500 m²/g, preferably from 200 to 450 m²/g, more preferably from 250 to 400 m²/g, and most preferably from 300 to 350 m²/g.

The surface area of the silica particles is typically measured as described above in relation to the first embodiment.

In a third embodiment, the present invention provides a chromatographic separation process for recovering a polyunsaturated fatty acid (PUFA) product from a feed mixture, which comprises introducing the feed mixture into a chromatography apparatus comprising one or more chromatographic columns containing:
(a) a liquid eluent phase which is an aqueous organic solvent; and
(b) a solid adsorbent phase which is a C18-bonded silica having a surface area of 500 m²/g or less,
wherein the pressure within the one or more chromatographic columns is less than 20 bar, thereby resulting in purification of the feed mixture.

It has been surprisingly found by the present inventors that silica having a surface area in this range provides an improved resolution between the PUFA peaks in the separation of a PUFA product from a feed mixture.

In the third embodiment, the silica preferably has a surface area of less than 450 m²/g, more preferably less than 400 m²/g, and most preferably less than 350 m²/g. The silica typically has a surface area of greater than 100 m²/g, preferably greater than 150 m²/g, more preferably greater than 200 m²/g, still more preferably greater than 250 m²/g, and most preferably greater than 300 m²/g. Thus, the silica typically has a surface area of from 100 to 500 m²/g, preferably from 200 to 450 m²/g, more preferably from 250 to 400 m²/g, and most preferably from 300 to 350 m²/g.

The surface area of the silica particles is typically measured as described above in relation to the first embodiment.

In the third embodiment, typically the average particle diameter is from 230 to 270 μm, preferably from 235 to 265 μm, more preferably from 240 to 260 μm, still more preferably from 245 to 260 μm, and most preferably from 250 to 260 μm.

In the third embodiment, typically the Dv(10) value of the silica is 160 μm or greater, preferably 165 μm or greater, more preferably 170 μm or greater, even more preferably 175 μm or greater, yet more preferably 180 μm or greater, and most preferably 185 μm or greater. Typically, the silica has a Dv(10) which is 225 μm or less, preferably 220 μm or less, more preferably 215 μm or less, and most preferably 210 μm or less. Thus, typically, the silica has a Dv(10) of from 160 to 225 μm, preferably from 170 to 220 μm, more preferably from 180 to 215 μm, and most preferably from 185 to 210 μm.

Thus, in the third embodiment, the silica typically has an average particle diameter of from 230 to 270 μm and a Dv(10) of 160 μm or greater, and preferably has an average particle diameter of from 235 to 265 μm and a Dv(10) of 165 μm or greater, and more preferably has an average particle diameter of from 240 to 260 μm and a Dv(10) of 175 μm or greater, and even more preferably has an average particle diameter of from 245 to 260 μm and a Dv(10) of 180 μm or greater, and most preferably has an average particle diameter of from 250 to 260 μm and a Dv(10) of 185 μm or greater.

Thus, in the third embodiment, the silica typically has an average particle diameter of from 230 to 270 μm and a Dv(10) of 225 μm or less, and preferably has an average particle diameter of from 235 to 265 μm and a Dv(10) of 225 μm or less, and more preferably has an average particle diameter of from 240 to 260 μm and a Dv(10) of 220 μm or less, and even more preferably has an average particle diameter of from 245 to 260 μm and a Dv(10) of 215 μm or less, and most preferably has an average particle diameter of from 250 to 260 μm and a Dv(10) of 210 μm or less.

Thus, in the third embodiment, the silica typically has an average particle diameter of from 230 to 270 μm and a Dv(10) of from 160 to 225 μm, preferably has an average particle diameter of from 235 to 265 μm and a Dv(10) of from 165 to 225 μm, and more preferably has an average particle diameter of from 240 to 260 μm and a Dv(10) of from 175 to 220 μm, and even more preferably has an average particle diameter of from 245 to 260 μm and a Dv(10) of from 180 to 215 μm, and most preferably has an average particle diameter of from 250 to 260 μm and a Dv(10) of from 185 to 210 μm.

The average particle diameter and the Dv(10) value are both typically measured as described above in relation to the first embodiment.

In the third embodiment, the silica typically has a carbon loading (% C) of from 15 to 24 wt %, preferably from 16 to 22 wt %, more preferably from 16.5 to 20 wt %, still more preferably from 17 to 19 wt %, and most preferably from 17.5 to 18 wt %, for example about 17.8 wt % or 17.9 wt %. Typically, substantially all of the carbon content derives from the C18-functionalisation of the silica particles.

The carbon loading of the silica particles is typically measured as described above in relation to the first embodiment.

In a preferred embodiment, the silica has the features of the second embodiment, in addition to those of the first and/or third embodiments. Thus, in this preferred embodiment, the present invention provides a chromatographic separation process for recovering a polyunsaturated fatty acid (PUFA) product from a feed mixture, which comprises introducing the feed mixture into a chromatography apparatus comprising one or more chromatographic columns containing:
(a) a liquid eluent phase which is an aqueous organic solvent; and
(b) a solid adsorbent phase which is a C18-bonded silica with a carbon loading of from 15 to 24 wt %,
wherein the pressure within the one or more chromatographic columns is less than 20 bar, thereby resulting in purification of the feed mixture, and further wherein:
(1) the silica has an average particle diameter of from 230 to 270 μm and a Dv(10) of 160 μm or greater; and/or
(2) the silica has a surface area of 500 m²/g or less.

In this preferred embodiment, the silica may have the features of the first and second embodiments. Thus, the silica typically has an average particle diameter of from 230 to 270 μm and a Dv(10) of 160 μm or greater, and a carbon loading of from 15 to 24 wt %. Preferably, the silica has an average particle diameter of from 235 to 265 μm and a Dv(10) of 165 μm or greater, and a carbon loading of from 16 to 22 wt %. More preferably, the silica has an average particle diameter of from 240 to 260 μm and a Dv(10) of 175 μm or greater, and a carbon loading of from 16.5 to 20 wt %. Still more preferably, the silica has an average particle diameter of from 245 to 260 μm and a Dv(10) of 180 μm or greater, and a carbon loading of from 17 to 19 wt %. Most preferably, the silica has an average particle diameter of from 250 to 260 μm and a Dv(10) of 185 μm or greater, and a carbon loading of from 17.5 to 18.0 wt %.

In this preferred embodiment where the silica has the features of the first and second embodiments, typically the silica has an average particle diameter of from 230 to 270 μm and a Dv(10) of 225 μm or less, and a carbon loading of from 15 to 24 wt %. Preferably, the silica has an average particle diameter of from 235 to 265 μm and a Dv(10) of 225 μm or less, and a carbon loading of from 16 to 22 wt %. More preferably, the silica has an average particle diameter of from 240 to 260 μm and a Dv(10) of 220 μm or less, and a carbon loading of from 16.5 to 20 wt %. Still more preferably, the silica has an average particle diameter of from 245 to 260 μm and a Dv(10) of 215 μm or less, and a carbon loading of from 17 to 19 wt %. Most preferably, the silica has an average particle diameter of from 250 to 260 μm and a Dv(10) of 210 μm or less, and a carbon loading of from 17.5 to 18.0 wt %.

In this preferred embodiment where the silica has the features of the first and second embodiments, typically the silica has an average particle diameter of from 230 to 270 μm and a Dv(10) of from 160 to 225 μm, and a carbon loading of from 15 to 24 wt %. Preferably, the silica has an average particle diameter of from 235 to 265 μm and a Dv(10) of from 165 to 225 μm, and a carbon loading of from 16 to 22 wt %. More preferably, the silica has an average particle diameter of from 240 to 260 μm and a Dv(10) of from 175 to 220 μm, and a carbon loading of from 16.5 to 20 wt %. Still more preferably, the silica has an average particle diameter of from 245 to 260 μm and a Dv(10) of from 180 to 215 μm, and a carbon loading of from 17 to 19 wt %. Most preferably, the silica has an average particle diameter of from 250 to 260 μm and a Dv(10) of from 185 to 210 μm, and a carbon loading of from 17.5 to 18.0 wt %.

Alternatively in this preferred embodiment, the silica has the features of the second and third embodiments. Thus, the silica typically has a carbon loading of from 15 to 24 wt %, and a total surface area of less than 500 m²/g. Preferably, the silica has a carbon loading of from 16 to 22 wt %, and a total surface area of less than 450 m²/g. More preferably, the silica has a carbon loading of from 16.5 to 20 wt %, and a total surface area of less than 400 m²/g. Still more preferably, the silica has a carbon loading of from 17 to 19 wt %, and a total surface area of less than 400 m²/g. Most preferably, the silica has a carbon loading of from 17.5 to 18.0 wt %, and a total surface area of less than 350 m²/g.

In this preferred embodiment where the silica has the features of the second and third embodiments, typically the silica has a carbon loading of from 15 to 24 wt %, and a total surface area of greater than 100 m²/g. Preferably, the silica has a carbon loading of from 16 to 22 wt %, and a total surface area of greater than 150 m²/g. More preferably, the silica has a carbon loading of from 16.5 to 20 wt %, and a total surface area of greater than 200 m²/g. Still more preferably, the silica has a carbon loading of from 17 to 19 wt %, and a total surface area of greater than 250 m²/g. Most preferably, the silica has a carbon loading of from 17.5 to 18.0 wt %, and a total surface area of greater than 300 m²/g.

In this preferred embodiment where the silica has the features of the second and third embodiments, typically the silica has a carbon loading of from 15 to 24 wt %, and a total surface area of from 100 to 500 m²/g. Preferably, the silica has a carbon loading of from 16 to 22 wt %, and a total surface area of from 150 to 450 m²/g. More preferably, the silica has a carbon loading of from 16.5 to 20 wt %, and a total surface area of from 200 to 450 m²/g. Still more preferably, the silica has a carbon loading of from 17 to 19 wt %, and a total surface area of from 250 to 400 m²/g. Most preferably, the silica has a carbon loading of from 17.5 to 18.0 wt %, and a total surface area of from 300 to 350 m²/g.

In a particularly preferred embodiment, the silica has all the features of the first, second and third embodiments. Thus, in this particularly preferred embodiment, typically the silica has an average particle diameter of from 230 to 270 μm and a Dv(10) of 160 μm or greater, a carbon loading of from 15 to 24 wt %, and a total surface area of less than 500 m²/g. Preferably, the silica has an average particle diameter of from 235 to 265 μm and a Dv(10) of 165 μm or greater, a carbon loading of from 16 to 22 wt %, and a total surface area of less than 450 m²/g. More preferably, the silica has an average particle diameter of from 240 to 260 μm and a Dv(10) of 175 μm or greater, a carbon loading of from 16.5 to 20 wt %, and a total surface area of less than 400 m²/g. Still more preferably, the silica has an average particle diameter of from 245 to 260 μm and a Dv(10) of 180 μm or greater, a carbon loading of from 17 to 19 wt %, and a total surface area of less than 400 m²/g. Most preferably, the silica has an average particle diameter of from 250 to 260 μm and a Dv(10) of 185 μm or greater, a carbon loading of from 17.5 to 18.0 wt %, and a total surface area of less than 350 m²/g.

In this particularly preferred embodiment, typically the silica has an average particle diameter of from 230 to 270 μm and a Dv(10) of 225 μm or less, a carbon loading of from 15 to 24 wt %, and a total surface area of greater than 100 m²/g. Preferably, the silica has an average particle diameter of from 235 to 265 μm and a Dv(10) of 225 μm or less, a carbon loading of from 16 to 22 wt %, and a total surface area of greater than 150 m²/g. More preferably, the silica has an average particle diameter of from 240 to 260 μm and a Dv(10) of 220 μm or less, a carbon loading of from 16.5 to 20 wt %, and a total surface area of greater than 200 m²/g. Still more preferably, the silica has an average particle diameter of from 245 to 260 μm and a Dv(10) of 215 μm or less, a carbon loading of from 17 to 19 wt %, and a total surface area of greater than 250 m²/g. Most preferably, the silica has an average particle diameter of from 250 to 260 μm and a Dv(10) of 210 μm or less, a carbon loading of from 17.5 to 18.0 wt %, and a total surface area of greater than 300 m²/g.

In this particularly preferred embodiment, typically the silica has an average particle diameter of from 230 to 270 μm and a Dv(10) of from 160 to 225 μm, a carbon loading of from 15 to 24 wt %, and a total surface area of from 100 to 500 m²/g. Preferably, the silica has an average particle diameter of from 235 to 265 μm and a Dv(10) of from 165 to 225 μm, a carbon loading of from 16 to 22 wt %, and a total surface area of from 150 to 450 m²/g. More preferably, the silica has an average particle diameter of from 240 to 260 μm and a Dv(10) of from 175 to 220 μm, a carbon loading of from 16.5 to 20 wt %, and a total surface area of from 200 to 450 m²/g. Still more preferably, the silica has an average particle diameter of from 245 to 260 μm and a Dv(10) of from 180 to 215 μm, a carbon loading of from 17 to 19 wt %, and a total surface area of from 250 to 400 m²/g. Most preferably, the silica has an average particle diameter of from 250 to 260 μm and a Dv(10) of from 185 to 210 μm, a carbon loading of from 17.5 to 18.0 wt %, and a total surface area of from 300 to 350 m²/g.

In any of the foregoing embodiments (i.e. in any of the first, second or third embodiments including any typical, preferred or particularly preferred sub-embodiments thereof), the silica particles are typically porous. Typically, the average pore diameter of the silica particles is from 60 to 200 Å. Preferably, the average pore diameter is from 65 to 160 Å, more preferably from 70 to 140 Å, still more preferably from 80 to 130 Å, yet more preferably from 90 to 120 Å, and most preferably from 95 to 115 Å, for example about 100 Å or about 110 Å.

In any of the foregoing embodiments, typically the silica particles have a total pore volume of 0.5 to 1.5 cc/g. In one preferable embodiment, the pore volume is from 0.6 to 0.84 cc/g, more preferably from 0.7 to 0.8 cc/g, and most preferably from 0.73 to 0.77 cc/g.

The total pore volume is typically measured by nitrogen gas adsorption, e.g. as described in “Characterization of Porous Solids and Powders: Surface Area, Pore Size and Density” by Lowell, Shields, Thomas and Thommes, Springer, Dordrecht, 2006 (pub: Springer), which is incorporated herein by reference in its entirety.

The average pore diameter d is calculated from the total pore volume V and surface area A using the equation d=4*V/A. This is the diameter of hypothetical uniform cylindrical pores having the same volume and area.

In any of the foregoing embodiments, typically the silica particles have a distinct particle size distribution such that at least 80% of the particles by number have a diameter of greater than 200 μm. Typically, at least 80% of the particles by number have a diameter of less than 500 μm. Typically, at least 80% of the particles by number have a diameter of from 200 to 500 μm. Preferably, at least 85% of the particles by number have a diameter of greater than 200 μm. Typically, at least 85% of the particles by number have a diameter of less than 500 μm. Typically, at least 85% of the particles by number have a diameter of from 200 to 500 μm. More preferably, at least 90% of the particles by number have a diameter of greater than 200 μm. Typically, at least 90% of the particles by number have a diameter of less than 500 μm. Typically, at least 90% of the particles by number have a diameter of from 200 to 500 μm. Most preferably, at least 95% of the particles by number have a diameter of greater than 200 μm. Typically, at least 95% of the particles by number have a diameter of less than 500 μm. Typically, at least 95% of the particles by number have a diameter of from 200 to 500 μm.

In any of the foregoing embodiments, typically the silica particles have a distinct particle size distribution such that at least 80% of the particles by volume have a diameter of greater than 200 μm. Typically, at least 80% of the particles by volume have a diameter of less than 500 μm. Typically, at least 80% of the particles by volume have a diameter of from 200 to 500 μm. Preferably, at least 85% of the particles by volume have a diameter of greater than 200 μm. Typically, at least 85% of the particles by volume have a diameter of less than 500 μm. Typically, at least 85% of the particles by volume have a diameter of from 200 to 500 μm. More preferably, at least 90% of the particles by volume have a diameter of greater than 200 μm. Typically, at least 90% of the particles by volume have a diameter of less than 500 μm. Typically, at least 90% of the particles by volume have a diameter of from 200 to 500 μm. Most preferably, at least 95% of the particles by volume have a diameter of greater than 200 μm. Typically, at least 95% of the particles by volume have a diameter of less than 500 μm. Typically, at least 95% of the particles by volume have a diameter of from 200 to 500 μm.

In any of the foregoing embodiments, typically the silica particles have a distinct particle size distribution such that at least 80% of the particles by mass have a diameter of greater than 200 μm. Typically, at least 80% of the particles by mass have a diameter of less than 500 μm. Typically, at least 80% of the particles by mass have a diameter of from 200 to 500 μm. Preferably, at least 85% of the particles by mass have a diameter of greater than 200 μm. Typically, at least 85% of the particles by mass have a diameter of less than 500 μm. Typically, at least 85% of the particles by mass have a diameter of from 200 to 500 μm. More preferably, at least 90% of the particles by mass have a diameter of greater than 200 μm. Typically, at least 90% of the particles by mass have a diameter of less than 500 μm. Typically, at least 90% of the particles by mass have a diameter of from 200 to 500 μm. Most preferably, at least 95% of the particles by mass have a diameter of greater than 200 μm. Typically, at least 95% of the particles by mass have a diameter of less than 500 μm. Typically, at least 95% of the particles by mass have a diameter of from 200 to 500 μm.

In any of the foregoing embodiments, typically the silica particles have a bulk (packed bed) density of 0.71 kg/dm³ or less, preferably 0.70 kg/dm³ or less, more preferably 0.69 kg/dm³ or less, still more preferably 0.68 kg/dm³ or less, even more preferably 0.67 kg/dm³ or less, and most preferably 0.66 kg/dm³ or less. Typically the silica particles have a bulk density of 0.4 kg/dm³ or greater, preferably 0.5 kg/dm³ or greater, more preferably 0.55 kg/dm³ or greater, still more preferably 0.60 kg/dm³ or greater, even more preferably 0.63 kg/dm³ or greater, and most preferably 0.65 kg/dm³ or greater.

Thus, in any of the foregoing embodiments, typically the silica particles have a bulk density of from 0.4 to 0.71 kg/dm³, preferably from 0.5 to 0.70 kg/dm³, more preferably from 0.55 to 0.69 kg/dm³, still more preferably from 0.60 to 0.68 kg/dm³, even more preferably from 0.63 to 0.67 kg/dm³, and most preferably from 0.65 to 0.66 kg/dm³.

In any of the foregoing embodiments, typically the Dv(90) value of the silica is 320 μm or greater. Dv(90) refers to the 90^{th} percentile of particle diameter in a plot of cumulative volume distribution of the silica particles against increasing particle diameter. The Dv(90) is sensitive to the number of coarse particles (i.e. particles of large size) present in a sample. Dv(90) can be determined by laser diffraction. Preferably, the Dv(90) value of the silica is 325 μm or greater, more preferably 330 μm or greater, and most preferably 335 μm or greater. Typically, the silica has a Dv(90) which is 390 μm or less, preferably 370 μm or less, more preferably 350 μm or less, and most preferably 340 μm or less. Thus, typically, the silica has a Dv(90) of from 320 to 290 μm, preferably from 325 to 370 μm, more preferably from 330 to 350 μm, and most preferably from 335 to 340 μm.

### The Liquid Eluent Phase

The process of the present invention uses an aqueous organic eluent in the chromatographic separation, i.e. a mixture of water and an organic solvent. Typically, the eluent is not a supercritical state. Typically, the eluent is a liquid.

Typically, the organic solvent is chosen from alcohols, ethers, esters, ketones and nitriles. Alcohols, ketones and nitriles are preferred.

Alcohol solvents are well known to the person skilled in the art. Alcohols are typically short chain alcohols. Alcohols typically are of formula ROH, wherein R is a straight or branched C₁-C₆ alkyl group. The C₁-C₆ alkyl group is preferably unsubstituted. Examples of alcohols include methanol, ethanol, n-propanol, i-propanol, n-butanol, i-butanol, s-butanol and t-butanol. Methanol and ethanol are preferred. Methanol is more preferred.

Ether solvents are well known to the person skilled in the art. Ethers are typically short chain ethers. Ethers typically are of formula R—O—R′, wherein R and R′ are the same or different and represent a straight or branched C₁-C₆ alkyl group. The C₁-C₆ alkyl group is preferably unsubstituted. Preferred ethers include diethylether, diisopropylether, and methyl t-butyl ether (MTBE).

Ester solvents are well known to the person skilled in the art. Esters are typically short chain esters. Esters typically are of formula R—(C═O)O—R′, wherein R and R′ are the same or different and represent a straight or branched C₁-C₆ alkyl group. Preferred esters include methylacetate and ethylacetate.

Ketone solvents are well known to the person skilled in the art. Ketones are typically short chain ketones. Ketones typically are of formula R—(C═O)—R′, wherein R and R′ are the same or different and represent a straight or branched C₁-C₆ alkyl group. The C₁-C₆ alkyl group is preferably unsubstituted. Preferred ketones include acetone, methylethylketone and methyl isobutyl ketone (MIBK).

Nitrile solvents are well known to the person skilled in the art. Nitriles are typically short chain nitriles. Nitriles typically are of formula R—CN, wherein R represents a straight or branched C₁-C₆ alkyl group. The C₁-C₆ alkyl group is preferably unsubstituted. Preferred nitriles include acetonitrile.

Typically, the organic solvent is miscible with water. Preferably, the organic solvent is chosen from tetrahydrofuran, isopropyl alcohol, n-propyl alcohol, methanol, ethanol, acetonitrile, 1,4-dioxane, N,N-dimethyl formamide, and dimethylsulfoxide. Methanol and acetonitrile are particularly preferred organic solvents.

The ratio between the organic solvent and water in the eluent is not particularly limited. However, typically the organic solvent:water ratio is from 99.9:0.1 to 75:25 parts by volume, preferably from 99.5:0.5 to 80:20 parts by volume. If the organic solvent is methanol, the methanol:water ratio is typically from 99.9:0.1 to 85:15 parts by volume, preferably from 99.5:0.5 to 88:12 parts by volume. If the organic solvent is acetonitrile, the acetonitrile:water ratio is typically from 99:1 to 75:25 parts by volume, preferably from 96:4 to 80:20 parts by volume.

### PUFA Products and Feed Mixtures

As used herein, the term “PUFA product” refers to a product comprising one or more polyunsaturated fatty acids (PUFAs), and/or derivatives thereof, typically of nutritional or pharmaceutical significance. Typically, the PUFA product is a single PUFA or derivative thereof. Alternatively, the PUFA product is a mixture of two or more PUFAs or derivatives thereof.

The term “polyunsaturated fatty acid” (PUFA) refers to fatty acids that contain more than one double bond. Such PUFAs are well known to the person skilled in the art. As used herein, a PUFA “derivative” is a PUFA in the form of a mono-, di- or tri-glyceride, ester, phospholipid, amide, lactone, or salt. Mono-, di- and triglycerides and esters are preferred. Triglycerides and esters are more preferred. Esters are even more preferred. Esters are typically alkyl esters, preferably C₁-C₆ alkyl esters, more preferably C₁-C₄ alkyl esters. Examples of esters include methyl and ethyl esters. Ethyl esters are most preferred.

Typically, the PUFA product is at least one ω-3 or ω-6 PUFA or a derivative thereof, preferably at least one ω-3 PUFA or a derivative thereof.

Examples of ω-3 PUFAs include eicosatrienoic acid (ETE), eicosatetraenoic acid (ETA), eicosapentaenoic acid (EPA), docosapentaenoic acid (DPA) and docosahexaenoic acid (DHA). EPA, DPA and DHA are preferred. EPA and DHA are most preferred.

Examples of ω-6 PUFAs include eicosadienoic acid, gamma-linolenic acid (GLA), dihomo-gamma-linolenic acid (DGLA), arachidonic acid (ARA), docosadienoic acid, adrenic acid and docosapentaenoic (ω-6) acid. ARA and GLA are preferred.

Preferably, the PUFA product is EPA, DHA, a derivative thereof or mixtures thereof. Typical derivatives include EPA and DHA mono-, di- and triglycerides and EPA and DHA esters, preferably alkyl esters such as C₁-C₄ alkyl esters.

More preferably, the PUFA product is EPA, DHA, or a derivative thereof. Typical derivatives include EPA and DHA mono-, di- and triglycerides and EPA and DHA esters, preferably alkyl esters such as C₁-C₄ alkyl esters.

Most preferably, the PUFA product is eicosapentaenoic acid (EPA), docosahexaenoic acid (DHA), EPA triglycerides, DHA triglycerides, EPA ethyl ester or DHA ethyl ester.

Particularly preferably, the PUFA product is EPA, DHA, EPA ethyl ester or DHA ethyl ester.

In one embodiment, the PUFA product is EPA and/or EPA ethyl ester (EE) In another embodiment, the PUFA product is DHA and/or DHA ethyl ester (EE).

In a yet further embodiment, the PUFA product is a mixture of EPA and DHA and/or EPA EE and DHA EE.

Typically, the PUFA product is obtained in a purity of greater than 80 GC-area %, preferably greater than 85 GC-area %, more preferably greater than 90 GC-area %, still more preferably greater than 95 GC-area %, yet more preferably greater than 97 GC-area %, even more preferably greater than 98 GC-area % and most preferably greater than 99 GC-area %, wherein “GC-area %” is the % of area under a gas chromatogram trace corresponding to the relevant product (here, the PUFA product). The GC-area % can typically be measured using the method for omega-3 fatty acid ethyl esters outlined in the European Pharmacopoeia 6.0, pages 2552-2554 (accessed at http://a_uspbpep com/ep60/omega-3. 420 etb: 1% 20ester 62090% 201250e.pdf), the contents of which are incorporated by reference herein in their entirety.

In a most preferred embodiment, the PUFA product is EPA or an EPA derivative, for example EPA ethyl ester, and is obtained at a purity greater than 90 GC-area %, preferably greater than 95 GC-area %, more preferably greater than 97 GC-area %, even more preferably greater than 98 GC-area %, still more preferably greater than 98.4 GC-area %. Preferably, the PUFA product is EPA or an EPA derivative, for example EPA ethyl ester, and is obtained at a purity between 98 and 99.5 GC-area %.

In an alternative preferred embodiment, the PUFA is DHA or a DHA derivative, for example DHA ethyl ester, and is obtained in a purity of greater than 80 GC-area %, preferably greater than 85 GC-area %, more preferably greater than 90 GC-area %, more preferably greater than 92 GC-area %, and most preferably greater than 95 GC-area %. Preferably, the PUFA product is DHA or an DHA derivative, for example EPA ethyl ester, and is obtained at a purity between 97 and 99.5 GC-area %.

Typically, in addition to said PUFA product, an additional secondary PUFA product is collected in the chromatographic separation process of the invention. Preferably, the PUFA product is EPA or a derivative thereof and the additional secondary PUFA product is DHA or a derivative thereof.

Typically, therefore, the process of the invention is configured to collect a PUFA product which is EPA or a derivative thereof. In such embodiments, a feed mixture is typically used which contains EPA, components which are more polar than EPA, and components which are less polar than EPA.

Alternatively, the process of the invention is configured to collect a PUFA product which is DHA or a derivative thereof. In such embodiments, a feed mixture is typically used which contains DHA, components which are more polar than DHA, and components which are less polar than DHA.

Alternatively, the process is configured to collect a PUFA product which is a concentrated mixture of EPA and DHA or derivatives thereof. In such embodiments, a feed mixture is used which contains EPA, DHA, components which are more polar than EPA and DHA, and components which are less polar than EPA and DHA.

Typically, the PUFA product contains 1 GC-area % or less, preferably 0.5 GC-area % or less, more preferably 0.25 GC-area % or less, still more preferably 0.1 GC-area % or less, and most preferably 0.01 GC-area % or less, of C18 fatty acid impurities, C18 fatty acid mono-, di- and triglyceride impurities and C18 fatty acid alkyl ester impurities. More typically, the PUFA product contains 1 GC-area % or less, preferably 0.5 GC-area % or less, more preferably 0.25 GC-area % or less, still more preferably 0.1 GC-area % or less, and most preferably 0.01 GC-area % or less, of impurities which are C18 fatty acids and derivatives thereof. Typical C18 fatty acid derivatives are as defined above for PUFA derivatives. As used herein, a C18 fatty acid is a C18 aliphatic monocarboxylic acid having a straight or branched hydrocarbon chain. Typical C18 fatty acids include stearic acid (C18:0), oleic acid (C18:1n9), vaccenic acid (C18:1n7), linoleic acid (C18:2n6), gamma-linolenic acid/GLA (C18:3n6), alpha-linolenic acid/ALA (C18:3n3) and stearidonic acid/SDA (C18:4n3). In certain preferred embodiments, the PUFA product is substantially free of the above-mentioned impurities.

Typically, the PUFA product is not a C18 PUFA, a C18 PUFA mono-, di- or triglyceride, or a C18 PUFA alkyl ester. More typically, the PUFA product is not a C18 PUFA or a C18 PUFA derivative. Typical C18 PUFAs include linoleic acid (C18:2n6), GLA (C18:3n6), and ALA (C18:3n3).

In the process of the present invention, the feed mixture is typically (i) a natural or synthetic feedstock comprising at least one PUFA product, or (ii) a partially purified feedstock comprising at least one PUFA product obtained from partial purification of a natural or synthetic feedstock. Thus, in one embodiment, the feed mixture is a natural or synthetic feedstock comprising at least one PUFA product. In another embodiment, the feed mixture is a partially purified feedstock comprising at least one PUFA product obtained from partial purification of a natural or synthetic feedstock.

Suitable feedstocks for separating by the process of the present invention may be obtained from natural sources including vegetable and animal oils and fats, and from synthetic sources including oils obtained from genetically modified plants, animals and micro-organisms including fungi and yeasts. Examples include fish oils, algal and microalgal oils and plant oils, for example borage oil, Echium oil and evening primrose oil. In one embodiment, the feed mixture is a fish oil. In another embodiment, the feed mixture is an algal oil. Algal oils and microalgal oils are particularly suitable when the desired PUFA product is EPA, ARA and/or DHA. Genetically modified yeast is particularly suitable when the desired PUFA product is EPA. Genetically modified plants are particularly suitable when the desired PUFA product is EPA, ARA and/or DHA.

In a particularly preferred embodiment the feedstock is a fish oil or fish-oil derived feedstock. It has advantageously been found that when a fish-oil or fish-oil derived feedstock is used, an EPA or EPA ethyl ester PUFA product can be produced by the process of the present invention in greater than 90 GC-area % purity, preferably greater than 95 GC-area % purity, more preferably greater than 97 GC-area % purity, even more preferably greater than 98 GC-area %, still more preferably greater than 98.4 GC-area %, for example between 98 and 99.5 GC-area %.

The feed mixtures typically contain the PUFA product and at least one more polar component and at least one less polar component. The less polar components have a stronger adherence to the adsorbent used in the process of the present invention than does the PUFA product. During operation, such less polar components typically move with the solid adsorbent phase in preference to the liquid eluent phase. The more polar components have a weaker adherence to the adsorbent used in the process of the present invention than does the PUFA product. During operation, such more polar components typically move with the liquid eluent phase in preference to the solid adsorbent phase. In embodiments of the present invention where the chromatographic separation step is carried out by actual or simulated moving bed chromatography, typically more polar components will be separated into a raffinate stream, and less polar components will be separated into an extract stream.

Examples of the more and less polar components include (1) other compounds occurring in natural oils (e.g. marine oils or vegetable oils), (2) by-products formed during storage, refining and previous concentration steps, and (3) contaminants from solvents or reagents which are utilized during previous concentration or purification steps.

Examples of (1) include: other unwanted PUFAs; saturated fatty acids; sterols, for example cholesterol; vitamins; and environmental pollutants, such as poly chlorobiphenyl (PCB), polyaromatic hydrocarbon (PAH) pesticides, chlorinated pesticides, dioxines and heavy metals. PCB, PAH, dioxines and chlorinated pesticides are all highly non-polar components.

Examples of (2) include isomers and oxidation or decomposition products from the PUFA product, for instance, auto-oxidation polymeric products of fatty acids or their derivatives.

Examples of (3) include urea, which may be added to remove saturated or mono-unsaturated fatty acids from the feed mixture.

Preferably, the feed mixture is a PUFA-containing marine oil (e.g. a fish oil), more preferably a marine oil (e.g. a fish oil) comprising EPA and/or DHA.

An example feed mixture for preparing concentrated EPA (EE) by the process of the present invention comprises 50-75% EPA (EE), 0 to 10% DHA (EE), and other components including other essential ω-3 and ω-6 fatty acids.

An example feed mixture for preparing concentrated EPA (EE) by the process of the present invention comprises 55% EPA (EE), 5% DHA (EE), and other components including other essential ω-3 and ω-6 fatty acids. DHA (EE) is less polar than EPA (EE).

An example feed mixture for preparing concentrated DHA (EE) by the process of the present invention comprises 50-75% DHA (EE), 0 to 10% EPA (EE), and other components including other essential ω-3 and ω-6 fatty acids.

An example feed mixture for preparing concentrated DHA (EE) by the process of the present invention comprises 75% DHA (EE), 7% EPA (EE) and other components including other essential ω-3 and ω-6 fatty acids. EPA (EE) is more polar than DHA (EE).

An example feed mixture for preparing a concentrated mixture of EPA (EE) and DHA (EE) by the process of the present invention comprises greater than 33% EPA (EE), and greater than 22% DHA (EE).

The feedstock may undergo chemical treatment before fractionation by the process of the present invention. For example, it may undergo glyceride transesterification or glyceride hydrolysis.

The feedstock may be partially purified before fractionation by the process of the present invention. For example, it may be purified by crystallisation, molecular distillation or fractional distillation, urea fractionation, extraction with silver nitrate or other metal salt solutions, iodolactonisation, supercritical fluid fractionation, or chromatography, preferably stationary bed chromatography or simulated or actual moving bed chromatography.

In some embodiments, the feedstock may undergo both chemical treatment and partial purification.

In other embodiments, a feedstock may be used directly as a feed mixture in the process of the present invention with no initial chemical treatment step and no partial purification.

Thus, in some embodiments of the present invention, the chromatographic separation process comprises introducing a feedstock as feed mixture directly into the chromatography apparatus. In these embodiments, the process may result in purification of the feed mixture to yield the PUFA product directly. Alternatively, the process may result in purification of the feed mixture to yield an intermediate product, wherein said intermediate product is subjected to further purification to obtain the PUFA product.

In other embodiments, the chromatographic separation process comprises introducing a chemically treated feedstock as feed mixture into chromatography apparatus. In these embodiments, the process may result in purification of the feed mixture to yield the PUFA product directly. Alternatively, the process may result in purification of the feed mixture to yield an intermediate product, wherein said intermediate product is subjected to further purification to obtain the PUFA product.

In other embodiments, the chromatographic separation process comprises introducing a partially purified feedstock as feed mixture into the chromatography apparatus. In these embodiments, the process may result in purification of the feed mixture to yield the PUFA product directly. Alternatively, the process may result in purification of the feed mixture to yield an intermediate product, wherein said intermediate product is subjected to further purification to obtain the PUFA product.

In other embodiments, the chromatographic separation process comprises introducing a partially purified and chemically treated feedstock as feed mixture into the chromatography apparatus. In these embodiments, the process may result in purification of the feed mixture to yield the PUFA product directly. Alternatively, the process may result in purification of the feed mixture to yield an intermediate product, wherein said intermediate product is subjected to further purification to obtain the PUFA product.

In any of the above embodiments in which the chromatographic separation process results in preparation of an intermediate product, in order to obtain the desired PUFA product from the intermediate product, further purification steps are carried out. Typically, said further purification is carried out in one or more chromatography apparatuses, preferably one or more stationary bed chromatography apparatuses or one or more actual or simulated moving bed chromatography apparatuses. More preferably, said further purification comprises flash column chromatography on one or more stationary bed chromatography apparatuses.

The PUFA product of the chromatographic separation process of the invention may be further treated either physically or chemically, e.g. by treating with bleaching earth or silica to reduce oxidation by-products, or by addition of an antioxidant (such as tocopherol).

In any embodiments of the present invention which involve two or more chromatographic separation steps in order to obtain the PUFA product, at least one of the chromatographic separation steps comprises a solid adsorbent as described herein. Preferably, each of the chromatographic separation steps comprises a solid adsorbent as described herein.

In one preferred embodiment, the chromatographic separation process involves two chromatographic separation steps in order to obtain the PUFA product. Preferably in this embodiment, both the first and second chromatographic separation steps are carried out in a chromatography apparatus as described herein, i.e. a chromatography apparatus operating at a pressure of 20 bar or less and comprising as the solid adsorbent phase a C18-bonded silica as described herein. However, alternatively only one of the first and/or second chromatographic separation steps is carried out in a chromatography apparatus as described herein, and the other separation step is carried out in a chromatography apparatus having a different solid adsorbent phase and/or operating pressure.

Thus, in this embodiment, typically the first chromatographic separation step is carried out in a chromatography apparatus as described herein, i.e. a chromatography apparatus operating at a pressure of 20 bar or less and comprising as the solid adsorbent phase a C18-bonded silica as described herein, and the second chromatographic step is carried out in a chromatography apparatus having a different solid adsorbent phase, e.g. an alternative silica, such as an alternative C18-bonded silica, a C8-bonded silica, pure silica, cyano-bonded silica and phenyl-bonded silica, or a non-silica based solid adsorbent phase. Alternatively, the first chromatographic separation step is carried out in a chromatography apparatus as described herein and the second chromatographic step is carried out in a chromatography apparatus operating at a pressure of greater than 20 bar. Alternatively, the first chromatographic separation step is carried out in a chromatography apparatus as described herein and the second chromatographic step is carried out in a chromatography apparatus operating at a pressure of greater than 20 bar having a different solid adsorbent phase, e.g. an alternative silica, such as an alternative C18-bonded silica, a C8-bonded silica, pure silica, cyano-bonded silica and phenyl-bonded silica, or a non-silica based solid adsorbent phase.

Alternatively, in this embodiment, the second chromatographic separation step may be carried out in a chromatography apparatus as described herein, i.e. a chromatography apparatus operating at a pressure of 20 bar or less and comprising as the solid adsorbent phase a C18-bonded silica as described herein, and the first chromatographic step is carried out in a chromatography apparatus having a different solid adsorbent phase, e.g. an alternative silica, such as an alternative C18-bonded silica, a C8-bonded silica, pure silica, cyano-bonded silica and phenyl-bonded silica, or a non-silica based solid adsorbent phase. Alternatively, the second chromatographic separation step is carried out in a chromatography apparatus as described herein and the first chromatographic step is carried out in a chromatography apparatus operating at a pressure of greater than 20 bar. Alternatively, the second chromatographic separation step is carried out in a chromatography apparatus as described herein and the first chromatographic step is carried out in a chromatography apparatus operating at a pressure of greater than 20 bar having a different solid adsorbent phase, e.g. an alternative silica, such as an alternative C18-bonded silica, a C8-bonded silica, pure silica, cyano-bonded silica and phenyl-bonded silica, or a non-silica based solid adsorbent phase.

Typically in this embodiment, the first and second chromatographic separation steps are carried out in different chromatography apparatuses. Alternatively, the first and second chromatographic separation steps are carried out in the same chromatography apparatus.

The chromatographic process of the present invention is typically configured such that the yield of PUFA product obtained from the feed mixture, based on the total mass of that PUFA product present in the feed mixture, is greater than 80 wt %, more preferably greater than 90 wt %, still more preferably greater than 95 wt %, and most preferably greater than 98 wt %.

### Operation of the Chromatographic Separation Process

Any known chromatography apparatus may be used for the chromatographic separation of the present invention. The number of chromatographic columns used in the separation is not particularly limited. The chromatography apparatus comprises one or more chromatographic columns, for example 1, 2, 3, 4, 5, 6, 7, 8, 9, or 10 columns. In some embodiments the number of columns is typically one. In other embodiments the number of columns is typically more than one, preferably 4 or more, more preferably 6 or more, even more preferably 8 or more, for example 4, 5, 6, 7, 8, 9, or 10 columns. Typically, there are no more than 25 columns, preferably no more than 20, more preferably no more than 15.

The dimensions of the columns used are not particularly limited, and will depend to some extent on the volume of feed mixture to be purified. A skilled person would easily be able to determine appropriately sized columns to use. The diameter of each column is typically between 50 and 5000 mm, preferably between 100 and 2500 mm, more preferably between 250 and 1500 mm, and most preferably between 250 and 1000 mm. The length of each column is typically between 10 and 300 cm, preferably between 10 and 200 cm, more preferably between 25 and 150 cm, even more preferably between 70 and 110 cm, and most preferably between 80 and 100 cm.

Typically, the process of the present invention is carried out at a maximum pressure of less than 20 bar. In some embodiments, the process of the present invention is carried out at a “medium” pressure of from 7 to 20 bar, preferably from 7.5 to 15 bar, and more preferably from 8 to 10 bar. Alternatively, in some embodiments, the process of the present invention is carried out at a “low” pressure of 7 bar or less, preferably from 1 to 7 bar, more preferably from 3 to 6 bar.

Typically, the process of the invention is carried out at room temperature, or a temperature greater than room temperature. Preferably, the process is carried out at a temperature greater than room temperature.

Typically, the temperature of at least one of the chromatographic columns through which the feed mixture is passed is greater than room temperature. More typically, the temperature of all of the chromatographic columns used is greater than room temperature.

As will be appreciated, if at least one chromatographic column is at a temperature greater than room temperature, it is the interior of the column which is important to the separation process.

Thus, it is typically the eluent and adsorbent inside the chromatographic column which may be at the temperature greater than room temperature. It is, of course, possible to achieve the required temperature inside the at least one chromatographic column by internal (for example by heating the eluent and/or feed mixture) and/or external means (for example by heating the outside of the chromatographic column by any known conventional means).

Typically, an elevated temperature can be achieved by heating the eluent and/or feed mixture. This has the effect of heating the columns internally.

Thus, the temperature of at least one of the chromatographic columns through which the feed mixture is passed can also be measured as the temperature of the eluent. Typically, therefore, the temperature of the eluent used in the chromatographic separation is greater than room temperature.

Alternatively, the required temperature of at least one of the chromatographic columns may be achieved by heating the columns. The heating may be carried out using, for example, an electric heating mantle, a heated water jacket or coil or by radiative heat lamps. The interior and/or exterior of the one or more chromatographic columns may typically be heated.

The required temperature of at least one of the chromatographic columns may be achieved by heating the columns and/or the aqueous organic solvent eluent, and/or the feed mixture.

Typically, the temperature greater than room temperature is greater than 30° C., preferably greater than 35° C., more preferably greater than 40° C., even more preferably greater than 45° C., even more preferably greater than 50° C., even more preferably greater than 55° C., and even more preferably greater than 57° C. A temperature of 56° C. is useful in certain embodiments.

Typically, the temperature greater than room temperature is up to 100° C., preferably up to 95° C., more preferably up to 90° C., even more preferably up to 85° C., even more preferably up to 80° C., even more preferably up to 75° C., and even more preferably up to 70° C.

Thus, typical temperature ranges are from 30 to 100° C., from 35 to 95° C., from 40 to 90° C., from 45 to 85° C., from 50 to 80° C., from 55 to 75° C. or from 57 to 70° C.

Preferred temperature ranges are from 40 to 70° C., preferably from 50 to 67° C., more preferably from 56 to 65° C., even more preferably from 57 to 63° C.

In certain embodiments a single chromatographic column may be used, preferably a single stationary chromatographic column. Separation in this manner is typically carried out using known stationary bed chromatography apparatuses. Separation in this manner may be referred to as “stationary bed” chromatography.

In other embodiments, more than one chromatographic column is used. This may involve passing the feed mixture through two or more chromatographic columns, which may be the same or different, arranged in series or in parallel. The number of columns used in this embodiment is not particularly limited, but typically does not exceed thirty columns.

One particular embodiment where multiple chromatographic columns are used is simulated or actual moving bed chromatography.

Simulated and actual moving bed chromatography apparatuses are well known to the person skilled in the art. Any known simulated or actual moving bed chromatography apparatus may be utilised for the purposes of the method of the present invention, as long as the apparatus is used in accordance with the process of the present invention. Those apparatuses described in U.S. Pat. Nos. 2,985,589, 3,696,107, 3,706,812, 3,761,533, FR-A-2103302, FR-A-2651148, FR-A-2651149, U.S. Pat. Nos. 6,979,402, 5,069,883 and 4,764,276 may all be used if configured in accordance with the process of the present invention. SMB processes as disclosed in, for example, WO 2011/080503, WO 2013/005046, WO 2013/005047, WO 2013/005048, WO 2013/005051, WO 2013/005052 and/or WO 2014/108686 may also be employed.

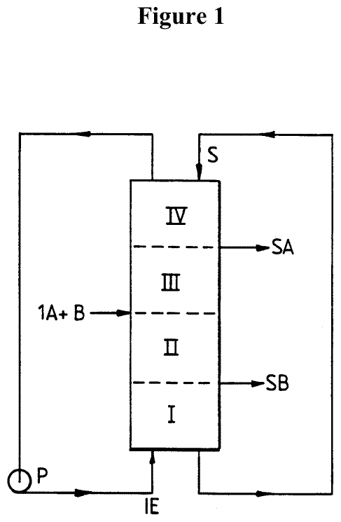

Typically, the chromatographic separation can involve the use of a single SMB separation step using conventional apparatus, such as for example depicted in FIG. 1. Separation in this manner may be referred to as “single pass” SMB.

In FIG. 1, counter-current contact between the liquid and solid phases is made in the column, which can be divided into four different zones.
Zone 1—Everything that is located between the eluant injection lines and the extract draw-off lines
Zone 2—Everything that is located between the extract draw-off lines and the feedstock injection lines
Zone 3—Everything that is located between the feedstock injection lines and the raffinate draw-off lines
Zone 4—Everything that is located between the raffinate draw-off lines and the eluant injection lines.

Because of the inlet/outlet flow rates, the liquid flow rate varies according to the zone, whereby Q_{I}, Q_{II}, Q_{III}, and Q_{IV} are the respective flow rates in zones I, II, III, and IV.

In one embodiment, the feedstock and eluent introduction points are periodically advanced downstream (in the direction of circulation of the main fluid), while the draw-off points for a raffinate and an extract are advanced simultaneously and according to the same increment (at least one column, for example).

Thus, in this embodiment, all of the inlet and output lines are moved simultaneously with each period ΔT and cycle time, at the end of which time they find their initial position is equal to Nc*ΔT, whereby Nc is the total number of columns.

In this embodiment, the inlet/outlet positions are moved simultaneously at fixed intervals. The position of the line is typically marked by line (n), which indicates that at a given moment a given inlet/outlet line is connected to the inlet of column n. For example, in a 12-column system, feedstock (9) means that the feedstock line is connected to the inlet of column 9, whereas raffinate (11) means that the raffinate line is connected to the inlet of column 11.

By using this definition, a system can be represented by: El(3)/Ext(6)/Feedstock(9)/Raff(11).

For this configuration, the number of columns in zones I, II, III, and IV are respectively: 3/3/2/4. The configuration of the system is then completely defined by:
Inlet/Outlet No. of Columns
At time 0: El(3)/Ext(6)/Feedstock(9)/Raff(11) 3/3/2/4
After a given time (the PERIOD), all of the inlet/outlet positions are moved simultaneously by one column, and the system is described as follows:
At time ΔT: El(4)/Ext(7)/Feedstock(10)/Raff(12) 3/3/2/4
After a new period, all of the positions will again be moved simultaneously by one column, and the system will then be described as follows:
At time 2*ΔT: El(5)/Ext(8)/Feedstock(11)/Raff(1) 3/3/2/4
At time 2*ΔT, the position of the raffinate has been moved from position 12 to position 1, which can also be written as position 13 modulo 12. ([13]12).

This presentation can be generalized to simulated moving beds that comprise a number of columns Nc. If, at a given moment, the configuration of the simulated moving bed is El(e)/Ext(x)/Feedstock(f)/Raff(r), simple reasoning makes it possible to find the number of columns that are contained in each zone:
Zone 1: Nb1=[x-e]_{Ne}; Zone 2: Nb2=[f-x]_{Ne}
Zone 3: Nb3=[r-f]_{Ne}; Zone 4: Nb4=[e-r]_{Ne}

The injection and draw-off points are shifted by one column after a period ΔT and by Nc columns after Nc periods. The number of columns in each zone remains unchanged. The injection and draw-off points therefore regain their initial positions after cycle time Nc*ΔT.

In another embodiment, the chromatographic separation can be carried out as described in U.S. Pat. No. 6,136,198 and Sa Gomes and Rodrigues, Chemical Engineering & Technology Special Issue: Preparative Chromatography and Downstream Processing, 2012, 35, 17-34, the entirety of which are incorporated herein by reference. These documents describe non-conventional methods of operating chromatographic separation processes. Typically in this embodiment, the process can be carried out, amongst others, using the so-called Varicol, Powerfeed, ISMB, Modicon, OSS or particle feed/partial discard methods. Preferably in this embodiment, the process is carried out using the Varicol method. This process is a single pass SMB process with asynchronous port switch, and can also be referred to as “asynchronous SMB”. The Varicol process is described in further detail below.

The “Varicol” embodiment differs from single pass SMB in the following respects:
(1) the zone lengths are not constant;
(2) the number of columns per zone is not constant over the period; and
(3) the inlet/outlet lines are not moved simultaneously.

In the “Varicol” embodiment, by not simultaneously moving the positions of the inlets and the outlets of fluid during the period and during the cycle time, it is possible to obtain improved results. Improvements include an increased purity of the product that is drawn off as an extract and as a raffinate, and a reduced cost of separation.

In the “Varicol” embodiment, at least one component of a mixture that contains it is purified in a device that has a set of chromatographic columns or chromatographic column sections that contain an adsorbent and are arranged in series and in a closed loop, whereby the loop comprises at least one feedstock injection point, a raffinate draw-off point, an eluent injection point, and an extract draw-off point, in which a chromatographic zone is determined by an injection point and a draw-off point or vice-versa, and at the end of a given period of time, all of the injection and draw-off points are shifted by one column or column section in a given direction that is defined relative to that of the flow of a main fluid that circulates through the loop, whereby the process is characterized in that during said period, the shifting of different injection and draw-off points of a column or column section is carried out at different times such that the lengths of the zones that are defined by said different points are variable.

The period is defined as the smallest time interval ΔT at the end of which each of the inlets and outlets has been shifted by one column or column section, whereby the shifting has not taken place simultaneously for all of the inlets and outlets. At the end of a cycle time Nc*ΔT the system has regained its initial position.

The adsorbent can in some aspects be selected from a molecular sieve, a zeolitic sieve, for example, that is used in the adsorption processes, or an adsorbent such as an ion-exchange resin. It may also be a stationary phase on a silica base, an inverse-phase adsorbent, and a chiral phase.

In the “Varicol” embodiment, it is possible to produce at least once the succession of following stages:
At moment t1 during said period ΔT, in a given direction, the position of the injection point or draw-off point is shifted relative to at least one zone by a column or column section, in such a way as to increase the length of said zone and to reduce the length of the zone that is adjacent to said zone, then at a moment t2 during said period, the position of an injection or draw-off point that is relative to at least one other zone is shifted in the same direction by a column or column section, in such a way as to increase the length of said other zone and to reduce the length of the zone that is adjacent to said other zone, and the operation is repeated if necessary such that after said time period ΔT the same column configuration as the initial configuration is regained with a shifting of all of the positions of the injection points and draw-off points of a column or a column section.

In one aspect of the “Varicol” embodiment, it is possible to continually vary the lengths of zones of a column, whereby the increase of one zone is compensated for by the reduction of the next zone (see, e.g., Table 2 of U.S. Pat. No. 6,136,198).

In another aspect of the “Varicol” embodiment, the increase in length of a zone can be compensated for by a reduction of the opposite zone (see, e.g., Table 3 of U.S. Pat. No. 6,136,198).

It is possible during the period to perform all of the shiftings of the injection or draw-off positions with an approximately constant time phase shift and advantageously with a time phase shift that is at least equal to a quarter-period.

In some aspects of the “Varicol” embodiment, it is possible to carry out during the period the shifting of the positions of the injection or draw-off points with a non-constant time phase shift.

In the “Varicol” embodiment, the flow rate of fluid that circulates in a given zone is generally kept approximately constant. It is also advantageous to carry out the shiftings of the positions of the injection and draw-off points in the same direction as that of the flow in the columns or column section. Further, at least one flow rate of fluid that circulates in an injection or draw-off line can be monitored by the pressure in the device. Preferably, it is the flow rate of the raffinate and/or the extract, whereby the other fluids are then under flow rate control.

It is advantageously possible in the “Varicol” embodiment to use a liquid as an eluent, but it is also possible to operate with a supercritical fluid or with a subcritical fluid.

The range of pressures in which the separations of products are carried out in the “Varicol” embodiment can be between 0.1 and 50 MPa and preferably between 0.5 and 30 MPa. The temperature in the columns is generally between 0° C. and 100° C. Typically, the number of columns or column sections is less than 8. For values of greater than 8, it is very advantageous to optimize the process by studying the influence of the number and the lengths of the columns in each zone that is combined at the moment of shifting during the period of the cycle.

The device used in the “Varicol” embodiment comprises a number of chromatographic columns or a chromatographic column section that contains an adsorbent, arranged in series and in a closed loop, whereby said loop comprises at least one pump for recirculating a fluid, a number of fluid injection lines in each column or column section that are connected to at least one injection pump and a number of fluid draw-off lines of each column or column section that are connected to at least one draw-off pump, at least one valve on each line, whereby said loop defines at least three chromatographic zones, whereby each of them is determined by a fluid injection point and a fluid draw-off point, whereby the device is characterized in that it comprises means for controlling the variation in time of the lengths of the zones that are connected to said valve and that are suitable for shifting by a column or column section the positions of the injection and draw-off points in an intermittent manner.

The valves that are used are advantageously all-or-none valves.

As an alternative to the processes described above, the chromatographic separation can involve the use of multiple SMB separations.

In one embodiment, the chromatographic separation can be carried out as described in WO 2011/080503 and WO 2013/005046, the entirety of which are incorporated herein by reference. Preferred process conditions specified in WO 2011/080503 and WO 2013/005046 are preferred process conditions for this embodiment, and may be incorporated from WO 2011/080503 and WO 2013/005046.

The process disclosed in WO 2011/080503 and WO 2013/005046 involves introducing an input stream to a simulated or actual moving bed chromatography apparatus having a plurality of linked chromatography columns containing, as eluent, an aqueous organic solvent, wherein the apparatus has a plurality of zones comprising at least a first zone and second zone, each zone having an extract stream and a raffinate stream from which liquid can be collected from said plurality of linked chromatography columns, and wherein (a) a raffinate stream containing the PUFA product together with more polar components is collected from a column in the first zone and introduced to a nonadjacent column in the second zone, and/or (b) an extract stream containing the PUFA product together with less polar components is collected from a column in the second zone and introduced to a nonadjacent column in the first zone, said PUFA product being separated from different components of the input stream in each zone. Separation in this manner may be referred to as a “double pass” SMB process.

In this “double pass” SMB process, the term “zone” refers to a plurality of linked chromatography columns containing, as eluent, an aqueous organic solvent, and having one or more injection points for an input stream, one or more injection points for water and/or organic solvent, a raffinate take-off stream from which liquid can be collected from said plurality of linked chromatography columns, and an extract take-off stream from which liquid can be collected from said plurality of linked chromatography columns. Typically, each zone has only one injection point for an input stream. In one embodiment, each zone has only one injection point for the aqueous organic solvent eluent. In another embodiment, each zone has two or more injection points for water and/or organic solvent.

The term “raffinate” is well known to the person skilled in the art. In the context of actual and simulated moving bed chromatography it refers to the stream of components that move more rapidly with the liquid eluent phase compared with the solid adsorbent phase. Thus, a raffinate stream is typically enriched with more polar components, and depleted of less polar components compared with an input stream.

The term “extract” is well known to the person skilled in the art. In the context of actual and simulated moving bed chromatography it refers to the stream of components that move more rapidly with the solid adsorbent phase compared with the liquid eluent phase. Thus, an extract stream is typically enriched with less polar components, and depleted of more polar components compared with an input stream.

As used herein, the term “nonadjacent” refers to columns, in for example the same apparatus, separated by one or more columns, preferably 3 or more columns, more preferably 5 or more columns, most preferably about 5 columns.

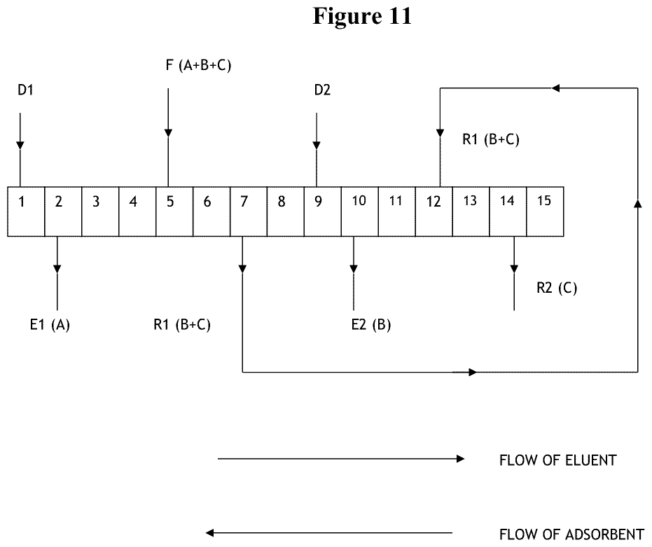

The “double pass” SMB process is illustrated in FIG. 11. An input stream F comprising the PUFA product (B) and more polar (C) and less polar (A) components is introduced into the top of column 5 in the first zone. Aqueous organic solvent desorbent is introduced into the top of column 1 in the first zone. In the first zone, the less polar components (A) are removed as extract stream E1 from the bottom of column 2. The PUFA product (B) and more polar components (C) are removed as raffinate stream R1 from the bottom of column 7. Raffinate stream R1 is then introduced into the second zone at the top of column 12. Aqueous organic solvent desorbent is introduced into the top of column 9 in the second zone. In the second zone, the more polar components (C) are removed as raffinate stream R2 at the bottom of column 14. The PUFA product (B) is collected as extract stream E2 at the bottom of column 10.

In this “double pass” SMB process, aqueous organic solvent is typically introduced into the top of column 1 in the first zone.

In this “double pass” SMB process, aqueous organic solvent is typically introduced into the top of column 9 in the second zone.

In this “double pass” SMB process, the input stream is typically introduced into the top of column 5 in the first zone.

In this “double pass” SMB process, a first raffinate stream is typically collected from the bottom of column 7 in the first zone and introduced into the top of column 12 in the second zone. The first raffinate stream may optionally be collected in a container before being introduced into column 12.

In this “double pass” SMB process, a first extract stream is typically removed from the bottom of column 2 in the first zone. The first extract stream may optionally be collected in a container and a portion reintroduced into the top of column 3 in the first zone. The rate of recycle of liquid collected via the extract stream from the first zone back into the first zone is the rate at which liquid is pumped from this container into the top of column 3.

In this “double pass” SMB process, a second raffinate stream is typically removed from the bottom of column 14 in the second zone.

In this “double pass” SMB process, a second extract stream is typically collected from the bottom of column 10 in the second zone. This second extract stream typically contains the PUFA product. The second extract stream may optionally be collected in a container and a portion reintroduced into the top of column 11 in the second zone. The rate of recycle of liquid collected via the extract stream from the second zone back into the second zone is the rate at which liquid is pumped from this container into the top of column 11.

In this “double pass” SMB process, the rate at which liquid collected via the extract stream from the first zone is recycled back into the first zone is typically faster than the rate at which liquid collected via the extract stream from the second zone is recycled back into the second zone. In this “double pass” SMB process, the eluent is typically substantially the same in each zone.

In this “double pass” SMB process, solvent can be recovered by evaporation, membrane or any other methods for people skilled in art for solvent recycle. The solvent, once completely, or partially, depleted from product or waste can be partially, or fully, recycled to the SMB as desorbent. Thus, in some aspects, concentration of the extract/raffinate stream (as the case may be) enriched in the product and/or the extract/raffinate stream (as the case may be) depleted of the product can be carried out by evaporation, drying or distillation.

Alternatively, concentration of the extract/raffinate stream (as the case may be) enriched in the product and/or the extract/raffinate stream (as the case may be) depleted of the product can be carried out by liquid extraction, membranes, crystallization, adsorption or other solvent recovery techniques.

Typically, at least one of the first and second chromatographic separation steps involves at least one, for example one, “double pass” SMB process as defined above.

In an alternative embodiment, the chromatographic separation can be carried out as described in WO 2013/005051 and/or WO 2013/005052, the entirety of which are incorporated herein by reference. Such embodiments involve:
(i) purifying an input stream in a first SMB step in a simulated or actual moving bed chromatography apparatus having a plurality of linked chromatography columns containing, as eluent, an aqueous organic solvent, to obtain a first product; and
(ii) purifying the first product obtained in (i) in a second SMB step using a simulated or actual moving bed chromatography apparatus having a plurality of linked chromatography columns containing, as eluent, an aqueous organic solvent, to obtain a second product; wherein
(a) the first and second SMB steps are carried out sequentially on the same chromatography apparatus, the first product being recovered between the first and second SMB steps and the process conditions in the chromatography apparatus being adjusted between the first and second SMB steps such that the PUFA product is separated from different components of the feed mixture in each SMB step; or
(b) the first and second SMB steps are carried out on separate first and second chromatography apparatuses respectively, the first product obtained from the first SMB step being introduced into the second chromatography apparatus, and the PUFA product being separated from different components of the feed mixture in each SMB step. Separation in this manner by be referred to as “back-to-back” SMB.

In this “back-to-back” SMB process, the term “simulated or actual moving bed chromatography apparatus” typically refers to a plurality of linked chromatography columns containing, as eluent, an aqueous organic solvent, and having one or more injection points for an input stream, one or more injection points for water and/or organic solvent, a raffinate take-off stream from which liquid can be collected from said plurality of linked chromatography columns, and an extract take-off stream from which liquid can be collected from said plurality of linked chromatography columns.

The chromatography apparatus used in this “back-to-back” SMB process has a single array of chromatography columns linked in series containing, as eluent, an aqueous organic solvent. Typically, each of the chromatography columns are linked to the two columns in the apparatus adjacent to that column. Thus, the output from a given column in the array is connected to the input of the adjacent column in the array, which is downstream with respect to the flow of eluent in the array. Thus, eluent can flow around the array of linked chromatography columns. Typically, none of the chromatography columns are linked to non-adjacent columns in the apparatus.

Typically, in this “back-to-back” SMB process, each apparatus has only one injection point for an input stream. In one embodiment, each apparatus has only one injection point for the aqueous organic solvent eluent. In another embodiment, each apparatus has two or more injection points for water and/or organic solvent.

The number of columns used in each apparatus in this “back-to-back” SMB process is not particularly limited. A skilled person would easily be able to determine an appropriate number of columns to use. The number of columns is typically 4 or more, preferably 6 or more, more preferably 8 or more, for example 4, 5, 6, 7, 8, 9, or 10 columns. In a preferred embodiment, 5 or 6 columns, more preferably 6 columns, are used. In another preferred embodiment, 7 or 8 columns, more preferably 8 columns are used. Typically, there are no more than 25 columns, preferably no more than 20, more preferably no more than 15.

In this “back-to-back” SMB process, the chromatographic apparatuses used in the first and second separation steps typically contain the same number of columns. For certain applications they may have different numbers of columns.

In this “back-to-back” SMB process, the columns in the chromatographic apparatuses used in the first and second SMB separation steps typically have identical dimensions but may, for certain applications, have different dimensions.

The flow rates to the columns are limited by maximum pressures across the series of columns and will depend on the column dimensions and particle size of the solid phases. One skilled in the art will easily be able to establish the required flow rate for each column dimension to ensure efficient desorption. Larger diameter columns will in general need higher flows to maintain linear flow through the columns.

In this “back-to-back” SMB process, for the typical column sizes outlined above, typically the flow rate of eluent into the chromatographic apparatus used in the first SMB separation step is from 50 to 300 L/min, preferably from 100 to 150 L/min. Typically, the flow rate of the extract from the chromatographic apparatus used in the first SMB separation step is from 5 to 150 L/min, preferably from 25 to 130 L/min. In embodiments where part of the extract from the first SMB separation step is recycled back into the apparatus used in the first SMB separation step, the flow rate of recycle is typically from 50 to 100 L/min, preferably about 75 L/min. Typically, the flow rate of the raffinate from the chromatographic apparatus used in the first SMB separation step is from 15 to 150 L/min, preferably from 20 to 125 L/min. In embodiments where part of the raffinate from the first SMB separation step is recycled back into the apparatus used in the first SMB separation step, the flow rate of recycle is typically from 20 to 75 L/min, preferably about 35 L/min. Typically, the flow rate of introduction of the input stream into the chromatographic apparatus used in the first SMB separation step is from 0.3 to 10 L/min, preferably from 0.5 to 7.5 L/min, more preferably from 1 to 4 L/min.

In this “back-to-back” SMB process, for the typical column sizes outlined above, typically the flow rate of eluent into the chromatographic apparatus used in the second SMB separation step is from 50 to 250 L/min, preferably from 100 to 225 L/min. Typically, the flow rate of the extract from the chromatographic apparatus used in the second SMB separation step is from 25 to 125 L/min, preferably from 50 to 120 L/min. In embodiments where part of the extract from the second SMB separation step is recycled back into the apparatus used in the second SMB separation step, the flow rate of recycle is typically from 40 to 90 L/min, preferably from 50 to 75 L/min, more preferably about 60 L/min. Typically, the flow rate of the raffinate from the chromatographic apparatus used in the second SMB separation step is from 25 to 150 L/min, preferably from 50 to 100 L/min, more preferably about 90 L/min. In embodiments where part of the raffinate from the second SMB separation step is recycled back into the apparatus used in the second SMB separation step, the flow rate of recycle is typically from 20 to 60 L/min, preferably about 30 L/min.

As the skilled person will appreciate, references to rates at which liquid is collected or removed via the various extract and raffinate streams refer to volumes of liquid removed in an amount of time, typically L/minute. Similarly, references to rates at which liquid is recycled back into an apparatus, typically to an adjacent column in the apparatus, refer to volumes of liquid recycled in an amount of time, typically L/minute.

In this “back-to-back” SMB process, actual moving bed chromatography is preferred.

The step time, i.e. the time between shifting the points of injection of the input stream and eluent, and the various take off points of the collected fractions, is not particularly limited, and will depend on the number and dimensions of the columns used, and the flow rate through the apparatus. A skilled person would easily be able to determine appropriate step times to use in the process of the present invention. The step time is typically from 100 to 1000 seconds, preferably from 200 to 800 seconds, more preferably from about 250 to about 750 seconds. In some embodiments, a step time of from 100 to 400 seconds, preferably 200 to 300 seconds, more preferably about 250 seconds, is appropriate. In other embodiments, a step time of from 600 to 900 seconds, preferably 700 to 800 seconds, more preferably about 750 seconds is appropriate.

The “back-to-back” SMB process comprises a first and second SMB separation step.

These two steps can easily be carried out on a single chromatographic apparatus. Thus, in one embodiment, (a) the first and second SMB separation steps are carried out sequentially on the same chromatography apparatus, the first product being recovered between the first and second SMB separation steps and the process conditions in the chromatography apparatus being adjusted between the first and second SMB separation steps such that the PUFA product is separated from different components of the input stream in each separation step. A preferred embodiment of this “back-to-back” SMB process is shown as FIG. 10A. Thus, the first SMB separation step (left hand side) is carried out on an SMB apparatus having 8 columns. Between the first and second SMB separation steps the first product is recovered in, for example, a container, the process conditions in the chromatography apparatus are adjusted such that the PUFA product is separated from different components of the input stream in each SMB separation step. The second SMB separation step (right hand side) is then carried out on the same SMB apparatus having 8 columns.

In embodiment (a), adjusting the process conditions typically refers to adjusting the process conditions in the apparatus as a whole, i.e. physically modifying the apparatus so that the conditions are different. It does not refer to simply reintroducing the first product back into a different part of the same apparatus where the process conditions might happen to be different.

Alternatively, first and second separate chromatographic apparatuses can be used in the first and second SMB separation steps. Thus, in another embodiment, (b) the first and second SMB separation steps are carried out on separate first and second chromatography apparatuses respectively, the first product obtained from the first SMB separation step being introduced into the second chromatography apparatus, and the PUFA product being separated from different components of the input stream in each SMB separation step.

In embodiment (b), the two SMB separation steps may either be carried out sequentially or simultaneously.

Thus, in embodiment (b) in the case where the two SMB separation steps are carried out sequentially, the first and second SMB separation steps are carried out sequentially on separate first and second chromatography apparatuses respectively, the first product being recovered between the first and second SMB separation steps and the process conditions in the first and second SMB chromatography apparatuses being adjusted such that the PUFA product is separated from different components of the input stream in each separation step. A preferred embodiment of this “back-to-back” SMB separation process is shown as FIG. 10B. Thus, the first SMB separation step (left hand side) is carried out on an SMB apparatus having 8 columns, one to eight. Between the first and second SMB separation steps the first product is recovered, for example in a container, and then introduced into a second separate SMB apparatus. The second SMB separation step (right hand side) is carried out on the second separate SMB apparatus which has 8 columns, nine to sixteen. The process conditions in the two chromatography apparatuses are adjusted such that the PUFA product is separated from different components of the input stream in each SMB separation step.

In embodiment (b) in the case where the two SMB separation steps are carried our simultaneously, the first and second SMB separation steps are carried out on separate first and second chromatography apparatuses respectively, the first product being introduced into the chromatography apparatus used in the second SMB separation step, and the process conditions in the first and second chromatography apparatuses being adjusted such that the PUFA product is separated from different components of the input stream in each SMB separation step. A preferred embodiment of this “back-to-back” SMB separation process is shown as FIG. 10c. Thus, the first SMB separation step (left hand side) is carried out on an SMB apparatus having 8 columns, one to eight. The first product obtained in the first SMB separation step is then introduced into the second separate chromatography apparatus used in the second SMB separation step. The first product may be passed from the first SMB separation step to the second SMB separation step directly or indirectly, for example via a container. The second SMB separation step (right hand side) is carried out on the second separate SMB apparatus which has 8 columns, nine to sixteen. The process conditions in the two chromatography apparatuses are adjusted such that the PUFA product is separated from different components of the input stream in each separation step.

In embodiment (b) in the case where the two SMB separation steps are carried our simultaneously, eluent circulates separately in the two separate chromatographic apparatuses. Thus, eluent is not shared between the two separate chromatographic apparatuses other than what eluent may be present as solvent in the first product which is purified in the second SMB separation step, and which is introduced into the chromatographic apparatus used in the second SMB separation step. Chromatographic columns are not shared between the two separate chromatographic apparatuses used in the first and second SMB separation steps.

In this “back-to-back” SMB process, after the first product is obtained in the first SMB separation step, the aqueous organic solvent eluent may be partly or totally removed before the first product is purified in the second SMB separation step. Alternatively, the first product may be purified in the second SMB separation step without the removal of any solvent present.

As mentioned above, in this “back-to-back” SMB process the PUFA product is separated from different components of the input stream in each SMB separation step. In embodiment (a), the process conditions of the single SMB apparatus used in both SMB separation steps are adjusted between the first and second SMB separation steps such that the PUFA product is separated from different components of the input stream in each separation step. In embodiment (b), the process conditions in the two separate chromatography apparatuses used in the first and second SMB separation steps are set such that the PUFA product is separated from different components of the input stream in each separation step.

Thus, in this “back-to-back” SMB process the process conditions in the first and second SMB separation steps vary. The process conditions which vary may include, for example, the size of the columns used, the number of columns used, the packing used in the columns, the step time of the SMB apparatus, the temperature of the apparatus, the water:organic solvent ration of the eluent used in the separation steps, or the flow rates used in the apparatus, in particular the recycle rate of liquid collected via the extract or raffinate streams.

Preferably in this “back-to-back” SMB process, the process conditions which may vary are the water:organic solvent ratio of the eluent used in the SMB separation steps, and/or the recycle rate of liquid collected via the extract or raffinate streams in the SMB separation steps. Both of these options are discussed in more detail below.

In this “back-to-back” SMB process, the first product obtained in the first SMB separation step is typically enriched in the PUFA product compared to the input stream.

In this “back-to-back” SMB process, the first product obtained in the first SMB separation step is then introduced into the chromatographic apparatus used in the second SMB separation step.

In this “back-to-back” SMB process, the first product is typically collected as the raffinate or extract stream from the chromatographic apparatus used in the first SMB separation process.

Typically in this “back-to-back” SMB process, the first product is collected as the raffinate stream in the first SMB separation step, and the second product is collected as the extract stream in the second SMB separation step. Thus, the raffinate stream collected in the first SMB separation step is used as the input stream in the second SMB separation step. The raffinate stream collected in the first SMB separation step typically contains the second product together with more polar components.

Alternatively in this “back-to-back” SMB process, the first product is collected as the extract stream in the first SMB separation step, and the second product is collected as the raffinate stream in the second SMB separation step. Thus, the extract stream collected in the first SMB separation step is used as the input stream in the second SMB separation step. The extract stream collected in the first SMB separation step typically contains the second product together with less polar components.

In this “back-to-back” SMB process the PUFA product is separated from different components of the input stream in each SMB separation step. Typically, the components separated in each SMB separation step of the process of the present invention have different polarities.

Preferably in this “back-to-back” SMB process, the PUFA product is separated from less polar components of the input stream in the first SMB separation step, and the PUFA product is separated from more polar components of the input stream in the second SMB separation step.

Typically in this “back-to-back” SMB process:
(a) part of the extract stream from the apparatus used in the first SMB separation step is recycled back into the apparatus used in the first SMB separation step; and/or
(b) part of the raffinate stream from the apparatus used in the first SMB separation step is recycled back into the apparatus used in the first SMB separation step; and/or
(c) part of the extract stream from the apparatus used in the second SMB separation step is recycled back into the apparatus used in the second SMB separation step; and/or
(d) part of the raffinate stream from the apparatus used in the second SMB separation step is recycled back into the apparatus used in the second SMB separation step.

Preferably in this “back-to-back” SMB process:
(a) part of the extract stream from the apparatus used in the first SMB separation step is recycled back into the apparatus used in the first SMB separation step; and
(b) part of the raffinate stream from the apparatus used in the first SMB separation step is recycled back into the apparatus used in the first SMB separation step; and
(c) part of the extract stream from the apparatus used in the second SMB separation step is recycled back into the apparatus used in the second SMB separation step; and
(d) part of the raffinate stream from the apparatus used in the second SMB separation step is recycled back into the apparatus used in the second SMB separation step.

The recycle in this “back-to-back” SMB process involves feeding part of the extract or raffinate stream out of the chromatography apparatus used in the first or second SMB separation step back into the apparatus used in that SMB step, typically into an adjacent column. This adjacent column is the adjacent column which is downstream with respect to the flow of eluent in the system.

In this “back-to-back” SMB process the rate at which liquid collected via the extract or raffinate stream in the first or second SMB separation steps is recycled back into the chromatography apparatus used in that SMB step is the rate at which liquid collected via that stream is fed back into the apparatus used in that SMB step, typically into an adjacent column, i.e. the downstream column with respect to the flow of eluent in the system.

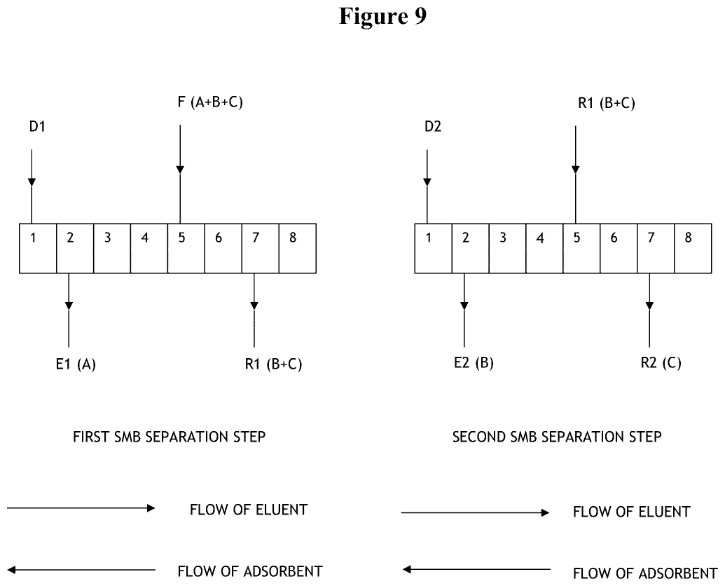

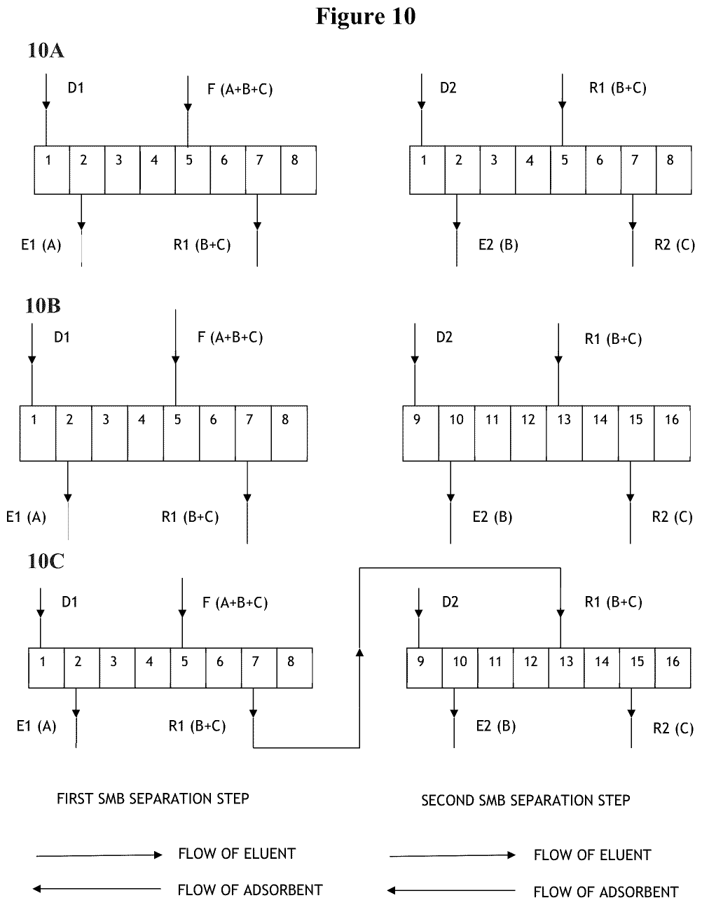

This can be seen with reference to FIG. 9. The rate of recycle of extract in the first SMB separation step is the rate at which extract collected from the bottom of column 2 of the chromatographic apparatus used in the first SMB separation step is fed into the top of column 3 of the chromatographic apparatus used in the first SMB separation step, i.e. the flow rate of liquid into the top of column 3 of the chromatographic apparatus used in the first SMB separation step.

In this “back-to-back” SMB process the rate of recycle of extract in the second SMB separation step is the rate at which extract collected at the bottom of column 2 of the chromatographic apparatus used in the second SMB separation step is fed into the top of column 3 of the chromatographic apparatus used in the second SMB separation step, i.e. the flow rate of liquid into the top of column 3 of the chromatographic apparatus used in the second SMB separation step.

In this “back-to-back” SMB process recycle of the extract and/or raffinate streams in the first and/or second SMB separation steps is typically effected by feeding the liquid collected via that stream in that SMB separation step into a container, and then pumping an amount of that liquid from the container back into the apparatus used in that SMB separation step, typically into an adjacent column. In this case, the rate of recycle of liquid collected via a particular extract or raffinate stream in the first and/or second SMB separation steps, typically back into an adjacent column, is the rate at which liquid is pumped out of the container back into the chromatography apparatus, typically into an adjacent column.

As the skilled person will appreciate, in this “back-to-back” SMB process the amount of liquid being introduced into a chromatography apparatus via the eluent and input streams is balanced with the amount of liquid removed from the apparatus, and recycled back into the apparatus.

Thus, in this “back-to-back” SMB process with reference to FIG. 9, for the extract stream, the flow rate of eluent (desorbent) into the chromatographic apparatus(es) used in the first and second SMB separation steps (D) is equal to the rate at which liquid collected via the extract stream in that SMB separation step accumulates in a container (E1 and E2) added to the rate at which extract is recycled back into the chromatographic apparatus used in that particular SMB separation step (D-E1 and D-E2).

In this “back-to-back” SMB process, for the raffinate stream from a SMB separation step, the rate at which extract is recycled back into the chromatographic apparatus used in that particular SMB separation step (D-E1 and D-E2) added to the rate at which feedstock is introduced into the chromatographic apparatus used in that particular SMB separation step (F and R1) is equal to the rate at which liquid collected via the raffinate stream in that particular SMB separation step accumulates in a container (R1 and R2) added to the rate at which raffinate is recycled back into the chromatographic apparatus used in that particular SMB separation step (D+F-El-R1 and D+R1-E2-R2).

In this “back-to-back” SMB process, the rate at which liquid collected from a particular extract or raffinate stream from a chromatography apparatus accumulates in a container can also be thought of as the net rate of removal of that extract or raffinate stream from that chromatography apparatus.

Typically in this “back-to-back” SMB process, the rate at which liquid collected via the extract and raffinate streams in the first SMB separation step is recycled back into the apparatus used in that separation step is adjusted such that the PUFA product can be separated from different components of the input stream in each SMB separation step.

Typically in this “back-to-back” SMB process, the rate at which liquid collected via the extract and raffinate streams in the second SMB separation step is recycled back into the apparatus used in that SMB separation step is adjusted such that the PUFA product can be separated from different components of the input stream in each SMB separation step.

Preferably in this “back-to-back” SMB process, the rate at which liquid collected via the extract and raffinate streams in each SMB separation step is recycled back into the apparatus used in that SMB separation step is adjusted such that the PUFA product can be separated from different components of the input stream in each SMB separation step.

Typically in this “back-to-back” SMB process, the rate at which liquid collected via the extract stream in the first SMB separation step is recycled back into the chromatography apparatus used in the first SMB separation step differs from the rate at which liquid collected via the extract stream in the second SMB separation step is recycled back into the chromatography apparatus used in the second SMB separation step, and/or the rate at which liquid collected via the raffinate stream in the first SMB separation step is recycled back into the chromatography apparatus used in the first SMB separation step differs from the rate at which liquid collected via the raffinate stream in the second SMB separation step is recycled back into the chromatography apparatus used in the second SMB separation step.

Varying the rate at which liquid collected via the extract and/or raffinate streams in the first or second SMB separation steps is recycled back into the apparatus used in that particular SMB separation step has the effect of varying the amount of more polar and less polar components present in the extract and raffinate streams. Thus, for example, a lower extract recycle rate results in fewer of the less polar components in that SMB separation step being carried through to the raffinate stream. A higher extract recycle rate results in more of the less polar components in that SMB separation step being carried through to the raffinate stream.

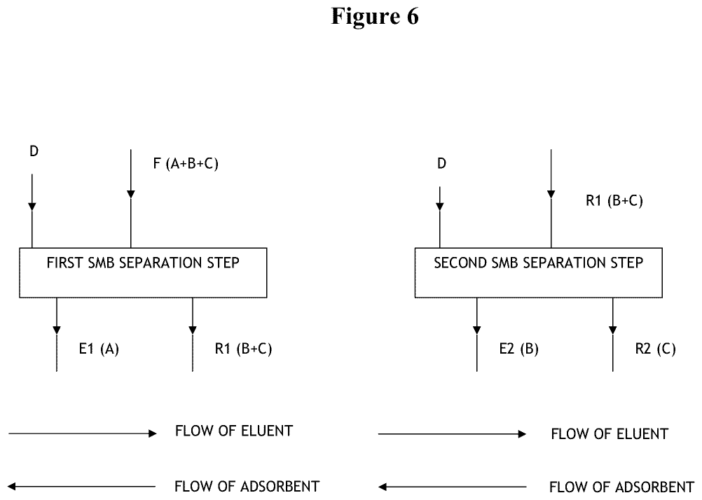

This can be seen, for example, in FIG. 6. The rate at which liquid collected via the extract stream in the first SMB separation step is recycled back into the chromatographic apparatus used in that SMB separation step (D-E1) will affect to what extent any of component A is carried through to the raffinate stream in the first SMB separation step (R1).

Typically in this “back-to-back” SMB process, the rate at which liquid collected via the extract stream in the first SMB separation step is recycled back into the chromatographic apparatus used in the first SMB separation step is faster than the rate at which liquid collected via the extract stream in the second SMB separation step is recycled back into the chromatographic apparatus used in the second SMB separation step. Preferably, a raffinate stream containing the second product together with more polar components is collected from the first SMB separation step and purified in a second SMB separation step, and the rate at which liquid collected via the extract stream in the first SMB separation step is recycled back into the chromatographic apparatus used in the first SMB separation step is faster than the rate at which liquid collected via the extract stream in the second SMB separation step is recycled back into the chromatographic apparatus used in the second SMB separation step.

Alternatively in this “back-to-back” SMB process, the rate at which liquid collected via the extract stream in the first SMB separation step is recycled back into the chromatographic apparatus used in the first SMB separation step is slower than the rate at which liquid collected via the extract stream in the second SMB separation step is recycled back into the chromatographic apparatus used in the second SMB separation step.

Typically in this “back-to-back” SMB process, the rate at which liquid collected via the raffinate stream in the first SMB separation step is recycled back into the chromatographic apparatus used in the first separation step is faster than the rate at which liquid collected via the raffinate stream in the second SMB separation step is recycled back into the chromatographic apparatus used in the second SMB separation step. Preferably, an extract stream containing the second product together with less polar components is collected from the first SMB separation step and purified in a second SMB separation step, and the rate at which liquid collected via the raffinate stream in the first SMB separation step is recycled back into the chromatographic apparatus used in the first SMB separation step is faster than the rate at which liquid collected via the raffinate stream in the second SMB separation step is recycled back into the chromatographic apparatus used in the second SMB separation step.

Alternatively in this “back-to-back” SMB process, the rate at which liquid collected via the raffinate stream in the first SMB separation step is recycled back into the chromatographic apparatus used in the first SMB separation step is slower than the rate at which liquid collected via the raffinate stream in the second SMB separation step is recycled back into the chromatographic apparatus used in the second SMB separation step.

In this “back-to-back” SMB process, where recycle rates are adjusted such that the PUFA product can be separated from different components of the input stream in each SMB separation step, the water:organic solvent ratio of the eluents used in each SMB separation step may be the same or different. Typical water:organic solvent ratios of the eluent in each SMB separation step are as defined above.

Typically in this “back-to-back” SMB process, the aqueous organic solvent eluent used in each SMB separation step has a different water:organic solvent ratio. The organic solvent used in each SMB separation step is the same. The water:organic solvent ratio used in each SMB separation step is preferably adjusted such that the PUFA product can be separated from different components of the input stream in each SMB separation step.

In this “back-to-back” SMB process, the eluting power of the eluent used in each of the SMB separation steps is typically different. Preferably, the eluting power of the eluent used in the first SMB separation step is greater than that of the eluent used in the second SMB separation step. In practice this is achieved by varying the relative amounts of water and organic solvent used in each SMB separation step.

Depending on the choice of organic solvent, they may be more powerful desorbers than water. Alternatively, they may be less powerful desorbers than water. Acetonitrile and alcohols, for example, are more powerful desorbers than water. Thus, when the aqueous organic solvent is aqueous alcohol or acetonitrile, the amount of alcohol or acetonitrile in the eluent used in the first SMB separation step is typically greater than the amount of alcohol or acetonitrile in the eluent used in the second SMB separation step.

Typically in this “back-to-back” SMB process, the water:organic solvent ratio of the eluent in the first SMB separation step is lower than the water:organic solvent ratio of the eluent in the second SMB separation step. Thus, the eluent in the first SMB separation step typically contains more organic solvent than the eluent in the second SMB separation step.

It will be appreciated that the ratios of water and organic solvent in each SMB separation step referred to above are average ratios within the totality of the chromatographic apparatus.

Typically in this “back-to-back” SMB process, the water:organic solvent ratio of the eluent in each SMB separation step is controlled by introducing water and/or organic solvent into one or more columns in the chromatographic apparatuses used in the SMB separation steps. Thus, for example, to achieve a lower water:organic solvent ratio in the first SMB separations step than in the second SMB separation step, water is typically introduced more slowly into the chromatographic apparatus used in the first SMB separation step than in the second SMB separation step.

Typically in this “back-to-back” SMB process, essentially pure organic solvent and essentially pure water may be introduced at different points in the chromatographic apparatus used in each SMB separation step. The relative flow rates of these two streams will determine the overall solvent profile in the chromatographic apparatus. Alternatively in this “back-to-back” SMB process, different mixtures of the organic solvent and water may be introduced at different points in each chromatographic apparatus used in each SMB separation step. That will involve introducing two or more different mixtures of the organic solvent and water into the chromatographic apparatus used in a particular SMB separation step, each organic solvent/water mixture having a different organic solvent:water ratio. The relative flow rates and relative concentrations of the organic solvent/water mixtures in this “back-to-back” SMB process will determine the overall solvent profile in the chromatographic apparatus used in that SMB separation step.

Preferably in this “back-to-back” SMB process, either:
(1) the first product containing the second product together with more polar components is collected as the raffinate stream in the first SMB separation step, and the second product is collected as the extract stream in the second SMB separation step; or
(2) the first product containing the second product together with less polar components is collected as the extract stream in the first SMB separation step, and the second product is collected as the raffinate stream in the second SMB separation step.

Option (1) is suitable for purifying EPA from an input stream.

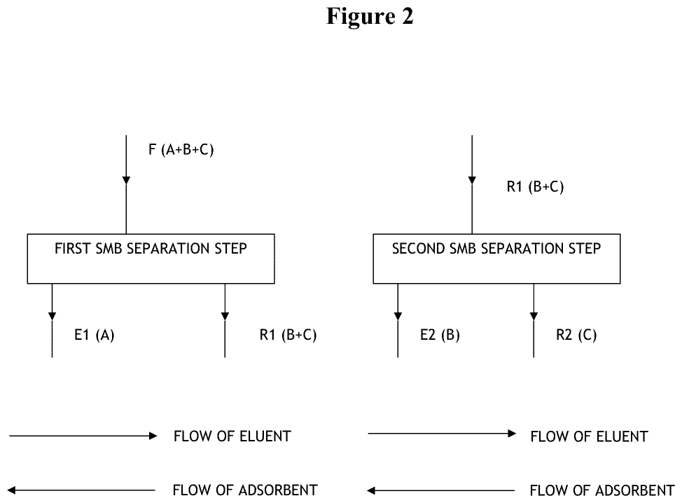

Option (1) is illustrated in FIG. 2. An input stream F comprising the second product (B) and more polar (C) and less polar (A) components is purified in the first SMB separation step. In the first SMB separation step, the less polar components (A) are removed as extract stream E1. The second product (B) and more polar components (C) are collected as raffinate stream R1. Raffinate stream R1 is the first product which is then purified in the second SMB separation step. In the second SMB separation step, the more polar components (C) are removed as raffinate stream R2. The second product (B) is collected as extract stream E2.

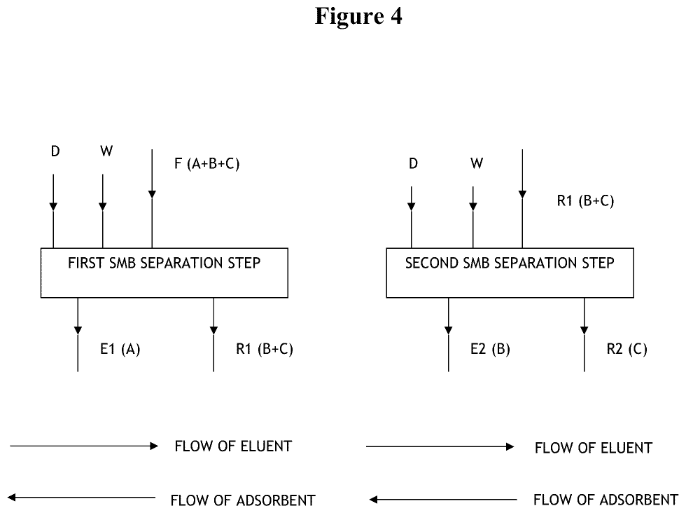

Option (1) is illustrated in more detail in FIG. 4. FIG. 4 is identical to FIG. 2, except that the points of introduction of the organic solvent desorbent (D) and water (W) into each chromatographic apparatus are shown. The organic solvent desorbent (D) and water (W) together make up the eluent. The (D) phase is typically essentially pure organic solvent, but may, in certain embodiments be an organic solvent/water mixture comprising mainly organic solvent. The (W) phase is typically essentially pure water, but may, in certain embodiments be an organic solvent/water mixture comprising mainly water, for example a 98% water/2% methanol mixture.

A further illustration of option (1) is shown in FIG. 6. Here there is no separate water injection point, and instead an aqueous organic solvent desorbent is injected at (D).

In option (1), the separation into raffinate and extract stream can be aided by varying the desorbing power of the eluent within each chromatographic apparatus. This can be achieved by introducing the organic solvent (or organic solvent rich) component of the eluent and the water (or water rich) component at different points in each chromatographic apparatus. Thus, typically, the organic solvent is introduced upstream of the extract take-off point and the water is introduced between the extract take-off point and the point of introduction of the feed into the chromatographic apparatus, relative to the flow of eluent in the system. This is shown in FIG. 4.

Typically, in option (1), the aqueous organic solvent eluent used in the first SMB separation step contains more organic solvent than the eluent used in the second SMB separation step, i.e. the water:organic solvent ratio in the first SMB separation step is lower than the water:organic solvent ratio in the second SMB separation step.

In option (1), the SMB separation can be aided by varying the rates at which liquid collected via the extract and raffinate streams in the first and second SMB separation steps is recycled back into the chromatographic apparatus used in that SMB separation step.

Typically, in option (1), the rate at which liquid collected via the extract stream in the first SMB separation step is recycled back into the chromatographic apparatus used in the first SMB separation step is faster than the rate at which liquid collected via the extract stream in the second SMB separation step is recycled back into the chromatographic apparatus used in the second SMB separation step.

In option (1) the first raffinate stream in the first SMB separation step is typically removed downstream of the point of introduction of the input stream into the chromatographic apparatus used in the first SMB separation step, with respect to the flow of eluent.

In option (1), the first extract stream in the first SMB separation step is typically removed upstream of the point of introduction of the input stream into the chromatographic apparatus used in the first SMB separation step, with respect to the flow of eluent.

In option (1), the second raffinate stream in the second SMB separation step is typically removed downstream of the point of introduction of the first product into the chromatographic apparatus used in the second SMB separation step, with respect to the flow of eluent.

In option (1), the second extract stream in the second SMB separation step is typically collected upstream of the point of introduction of the first product into the chromatographic apparatus used in the second SMB separation step, with respect to the flow of eluent.

Typically in option (1), the organic solvent or aqueous organic solvent is introduced into the chromatographic apparatus used in the first SMB separation step upstream of the point of removal of the first extract stream, with respect to the flow of eluent.

Typically in option (1), when water is introduced into the chromatographic apparatus used in the first SMB separation step, the water is introduced into the chromatographic apparatus used in the first SMB separation step upstream of the point of introduction of the input stream but downstream of the point of removal of the first extract stream, with respect to the flow of eluent.

Typically in option (1), the organic solvent or aqueous organic solvent is introduced into the chromatographic apparatus used in the second SMB separation step upstream of the point of removal of the second extract stream, with respect to the flow of eluent.

Typically in option (1), when water is introduced into the chromatographic apparatus used in the second SMB separation step, the water is introduced into the chromatographic apparatus used in the second SMB separation step upstream of the point of introduction of the first product but downstream of the point of removal of the second extract stream, with respect to the flow of eluent.

Option (2) is suitable for purifying DHA from an input stream.

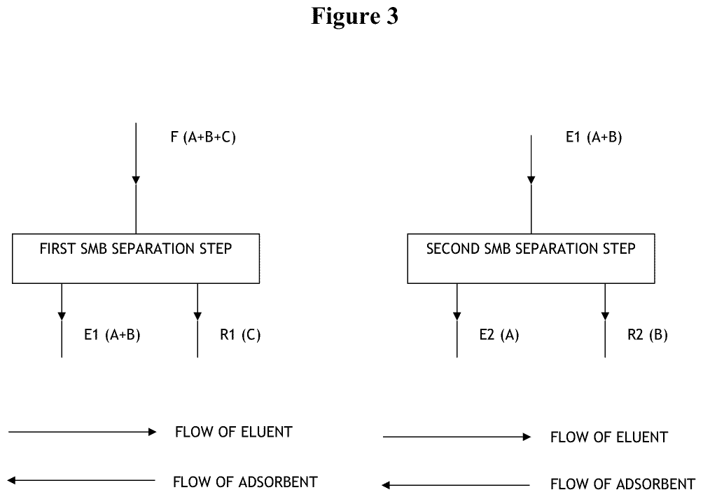

Option (2) is illustrated in FIG. 3. An input stream F comprising the second product (B) and more polar (C) and less polar (A) components is purified in the first SMB separation step.

In the first s SMB separation step, the more polar components (C) are removed as raffinate stream R1. The second product (B) and less polar components (A) are collected as extract stream E1. Extract stream E1 is the first product which is then purified in the second SMB separation step. In the second SMB separation step, the less polar components (A) are removed as extract stream E2. The second product (B) is collected as raffinate stream R2.

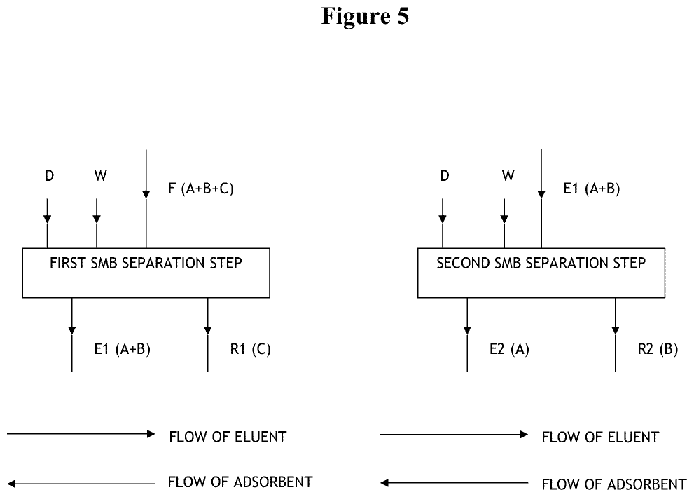

Option (2) is illustrated in more detail in FIG. 5. FIG. 5 is identical to FIG. 3, except that the points of introduction of the organic solvent desorbent (D) and water (W) into each chromatographic apparatus are shown. As above, the (D) phase is typically essentially pure organic solvent, but may, in certain embodiments be an organic solvent/water mixture comprising mainly organic solvent. The (W) phase is typically essentially pure water, but may, in certain embodiments be an organic solvent/water mixture comprising mainly water, for example a 98% water/2% methanol mixture.

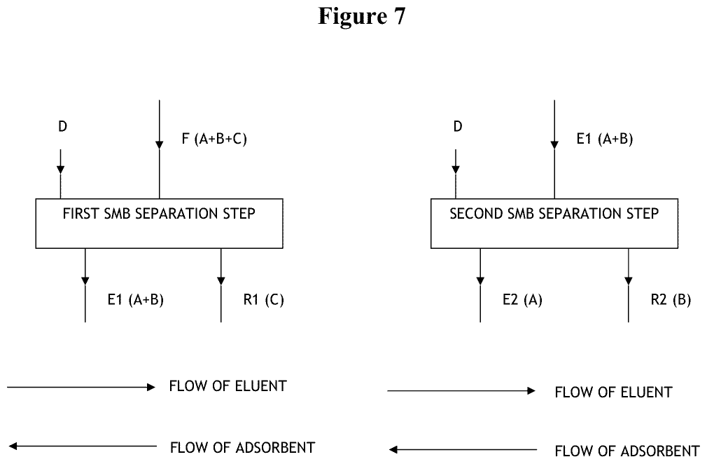

A further illustration of option (2) is shown in FIG. 7. Here there is no separate water injection point, and instead an aqueous organic solvent desorbent is injected at (D).

Typically in option (2), the rate at which liquid collected via the raffinate stream in the first SMB separation step is reintroduced into the chromatographic apparatus used in the first SMB separation step is faster than the rate at which liquid collected via the raffinate stream in the second SMB separation step is reintroduced into the chromatographic apparatus used in the second SMB separation step.

Typically in option (2), the aqueous organic solvent eluent used in the first SMB separation step contains less organic solvent than the eluent used in the second SMB separation step, i.e. the water:organic solvent ratio in the first SMB separation step is higher than in the second SMB separation step.

In option (2) the first raffinate stream in the first separation step is typically removed downstream of the point of introduction of the input stream into the chromatographic apparatus used in the first SMB separation step, with respect to the flow of eluent.

In option (2), the first extract stream in the first SMB separation step is typically removed upstream of the point of introduction of the input stream into the chromatographic apparatus used in the first SMB separation step, with respect to the flow of eluent.

In option (2), the second raffinate stream in the second SMB separation step is typically removed downstream of the point of introduction of the first product into the chromatographic apparatus used in the second SMB separation step, with respect to the flow of eluent.

In option (2), the second extract stream in the second SMB separation step is typically collected upstream of the point of introduction of the first product into the chromatographic apparatus used in the second SMB separation step, with respect to the flow of eluent.

Typically in option (2), the organic solvent or aqueous organic solvent is introduced into the chromatographic apparatus used in the first SMB separation step upstream of the point of removal of the first extract stream, with respect to the flow of eluent.

Typically in option (2), when water is introduced into the chromatographic apparatus used in the first SMB separation step, the water is introduced into the chromatographic apparatus used in the first SMB separation step upstream of the point of introduction of the input stream but downstream of the point of removal of the first extract stream, with respect to the flow of eluent.

Typically in option (2), the organic solvent or aqueous organic solvent is introduced into the chromatographic apparatus used in the second SMB separation step upstream of the point of removal of the second extract stream, with respect to the flow of eluent.

Typically in option (2), when water is introduced into the chromatographic apparatus used in the second SMB separation step, the water is introduced into the chromatographic apparatus used in the second SMB separation step upstream of the point of introduction of the first product but downstream of the point of removal of the second extract stream, with respect to the flow of eluent.

In this “back-to-back” SMB process, each of the simulated or actual moving bed chromatography apparatus used in the first and second SMB separation steps preferably consist of eight chromatographic columns. These are referred to as columns 1 to 8. In each apparatus the eight columns are arranged in series so that the bottom of column 1 is linked to the top of column 2, the bottom of column 2 is linked to the top of column 3 . . . etc., and the bottom of column 8 is linked to the top of column 1. These linkages may optionally be via a holding container, with a recycle stream into the next column. The flow of eluent through the system is from column 1 to column 2 to column 3 etc. The effective flow of adsorbent through the system is from column 8 to column 7 to column 6 etc.

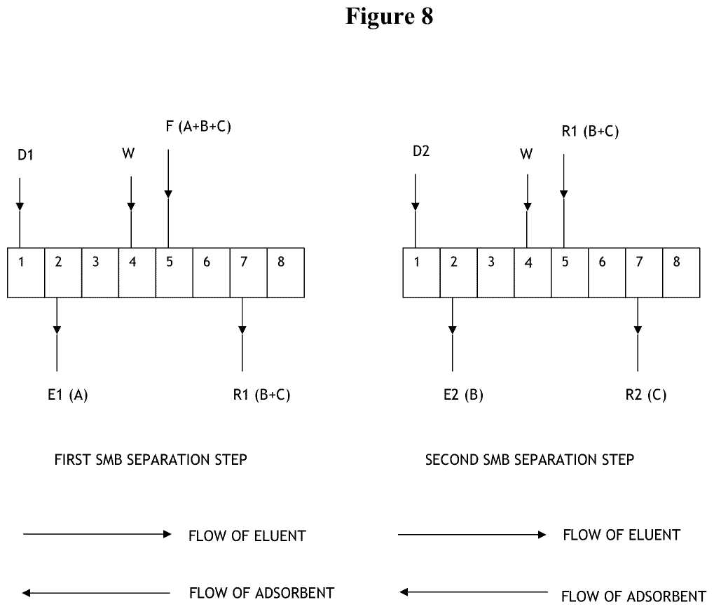

This is illustrated in FIG. 8. An input stream F comprising the second product (B) and more polar (C) and less polar (A) components is introduced into the top of column 5 in the chromatographic apparatus used in the first SMB separation step. Organic solvent desorbent is introduced into the top of column 1 of the chromatographic apparatus used in the first SMB separation step. Water is introduced into the top of column 4 of the chromatographic apparatus used in the first SMB separation step. In the first SMB separation step, the less polar components (A) are removed as extract stream E1 from the bottom of column 2. The second product (B) and more polar components (C) are removed as raffinate stream R1 from the bottom of column 7. Raffinate stream R1 is the first product which is then purified in the second SMB separation step, by being introduced into the chromatographic apparatus used in the second SMB separation step at the top of column 5. Organic solvent desorbent is introduced into the top of column 1 in the chromatographic apparatus used in the second SMB separation step. Water is introduced into the top of column 4 in the chromatographic apparatus used in the second SMB separation step. In the second SMB separation step, the more polar components (C) are removed as raffinate stream R2 at the bottom of column 7. The second product (B) is collected as extract stream E2 at the bottom of column 2.

In the “back-to-back” SMB process shown in FIG. 8, organic solvent is typically introduced into the top of column 1 of the chromatographic apparatus used in the first SMB separation step.

In the “back-to-back” SMB process shown in FIG. 8, water is typically introduced into the top of column 4 of the chromatographic apparatus used in the first SMB separation step.

In the “back-to-back” SMB process shown in FIG. 8, organic solvent is typically introduced into the top of column 1 of the chromatographic apparatus used in the second SMB separation step.

In the “back-to-back” SMB process shown in FIG. 8, organic solvent is typically introduced into the top of column 4 of the chromatographic apparatus used in the second SMB separation step.

In the “back-to-back” SMB process shown in FIG. 8, the input stream is typically introduced into the top of column 5 of the chromatographic apparatus used in the first SMB separation step.

In the “back-to-back” SMB process shown in FIG. 8, a first raffinate stream is typically collected as the first product from the bottom of column 7 of the chromatographic apparatus used in the first SMB separation step. This first product is then purified in the second SMB separation step and is typically introduced into the top of column 5 of the chromatographic apparatus used in the second SMB separation step. The first raffinate stream may optionally be collected in a container before being purified in the second SMB separation step.

In the “back-to-back” SMB process shown in FIG. 8, a first extract stream is typically removed from the bottom of column 2 of the chromatographic apparatus used in the first SMB separation step. The first extract stream may optionally be collected in a container and reintroduced into the top of column 3 of the chromatographic apparatus used in the first SMB separation step.

In the “back-to-back” SMB process shown in FIG. 8, a second raffinate stream is typically removed from the bottom of column 7 of the chromatographic apparatus used in the second SMB separation step.

In the “back-to-back” SMB process shown in FIG. 8, a second extract stream is typically collected from the bottom of column 2 of the chromatographic apparatus used in the second SMB separation step. This second extract stream typically contains the second product. The second extract stream may optionally be collected in a container and reintroduced into the top of column 3 of the chromatographic apparatus used in the second SMB separation step.

In the “back-to-back” SMB process shown in FIG. 8, the eluent used is typically as defined above.

Typically, in this “back-to-back” SMB process, the water:organic solvent ratio in the chromatographic apparatus used in the first SMB separation step is lower than the water:organic solvent ratio in the chromatographic apparatus used in the second SMB separation step. Thus, the eluent in the first SMB separation step typically contains more organic solvent than the eluent used in the second SMB separation step.

In this “back-to-back” SMB process, the water:organic solvent ratio in the first SMB separation step is typically from 0.5:99.5 to 1.5:98.5 parts by volume. The water:organic solvent ratio in the second SMB separation step is typically from 2:98 to 6:94 parts by volume.

In this “back-to-back” SMB process, although the apparatus of FIG. 8 is configured as shown in FIG. 10a, the configurations shown in FIGS. 10b and 10c could also be used.

This “back-to-back” SMB process is also illustrated in FIG. 9. An input stream F comprising the second product (B) and more polar (C) and less polar (A) components is introduced into the top of column 5 in the chromatographic apparatus used in the first SMB separation step. Aqueous organic solvent desorbent is introduced into the top of column 1 in the chromatographic apparatus used in the first SMB separation step. In the first SMB separation step, the less polar components (A) are removed as extract stream E1 from the bottom of column 2. The second product (B) and more polar components (C) are removed as raffinate stream R1 from the bottom of column 7. Raffinate stream R1 is the first product which is purified in the second SMB separation step by being introduced into the top of column 4 of the chromatographic apparatus used in the second SMB separation step. Aqueous organic solvent desorbent is introduced into the top of column 1 in the chromatographic apparatus used in the second SMB separation step. In the second SMB separation step, the more polar components (C) are removed as raffinate stream R2 at the bottom of column 7. The second product (B) is collected as extract stream E2 at the bottom of column 2.

In the “back-to-back” SMB process shown in FIG. 9, aqueous organic solvent is typically introduced into the top of column 1 in the chromatographic apparatus used in the first SMB separation step.

In the “back-to-back” SMB process shown in FIG. 9, aqueous organic solvent is typically introduced into the top of column 9 in the chromatographic apparatus used in the second SMB separation step.

In the “back-to-back” SMB process shown in FIG. 9, the input stream is typically introduced into the top of column 5 in the chromatographic apparatus used in the first SMB separation step.

In the “back-to-back” SMB process shown in FIG. 9, a first raffinate stream is typically collected as the first product from the bottom of column 7 of the chromatographic apparatus used in the first SMB separation step. This first product is then purified in the second SMB separation step and is typically introduced into the top of column 5 of the chromatographic apparatus used in the second SMB separation step. The first raffinate stream may optionally be collected in a container before being purified in the second SMB separation step.

In the “back-to-back” SMB process shown in FIG. 9, a first extract stream is typically removed from the bottom of column 2 of the chromatographic apparatus used in the first SMB separation step. The first extract stream may optionally be collected in a container and a portion reintroduced into the top of column 3 of the chromatographic apparatus used in the first SMB separation step. The rate of recycle of liquid collected via the extract stream in the first SMB separation step back into the chromatographic apparatus used in the first SMB separation step is the rate at which liquid is pumped from this container into the top of column 3.

In the “back-to-back” SMB process shown in FIG. 9, a second raffinate stream is typically removed from the bottom of column 7 of the chromatographic apparatus used in the first SMB separation step.

In the “back-to-back” SMB process shown in FIG. 9, a second extract stream is typically collected from the bottom of column 2 of the chromatographic apparatus used in the first SMB separation step. This second extract stream typically contains the second product. The second extract stream may optionally be collected in a container and a portion reintroduced into the top of column 3 of the chromatographic apparatus used in the first SMB separation step. The rate of recycle of liquid collected via the extract stream from the second SMB separation step back into the chromatographic apparatus used in the second SMB separation step is the rate at which liquid is pumped from this container into the top of column 3.

In the “back-to-back” SMB process shown in FIG. 9, the eluent used is as defined above.

Typically, in this “back-to-back” SMB process, the water:organic solvent ratio in the chromatographic apparatus used in the first SMB separation step is lower than the water:organic solvent ratio in the chromatographic apparatus used in the second SMB separation step. Thus, the eluent used in the first SMB separation step typically contains more organic solvent than the eluent used in the second SMB separation step.

In this “back-to-back” SMB process, the water:organic solvent ratio in the first SMB separation step is typically from 0.5:99.5 to 1.5:98.5 parts by volume. The water:organic solvent ratio in the second SMB separation step is typically from 2:98 to 6:94 parts by volume.

In this “back-to-back” SMB process, the rate at which liquid collected via the extract stream from the first SMB separation step is recycled back into the chromatographic apparatus used in the first SMB separation step is typically faster than the rate at which liquid collected via the extract stream from the second SMB separation step is recycled back into the chromatographic apparatus used in the second SMB separation step. In this case, the aqueous organic solvent eluent is typically substantially the same in each SMB separation step.

In this “back-to-back” SMB process, although the apparatus of FIG. 9 is configured as shown in FIG. 10a, the configurations shown in FIGS. 10b and 10c could also be used.

Typically, the chromatographic separation of the present invention involves at least one, for example one, “back-to-back” SMB process as defined above.

In one preferred embodiment, the chromatographic separation process of the present invention comprises two distinct chromatographic separation steps in order to obtain the desired PUFA product from a feed mixture. Thus, in this embodiment the chromatographic separation process comprises:
(i) purifying the feed mixture in a first chromatographic separation step using as eluent a mixture of water and a first organic solvent, to obtain an intermediate product; and
(ii) purifying the intermediate product in a second chromatographic separation step using as eluent a mixture of water and a second organic solvent, to obtain the PUFA product.

Typically in this embodiment, the second organic solvent is different from the first organic solvent. This so-called “mixed solvents” process is particularly effective at removing C18 impurities from the desired PUFA product, and is described in WO 2014/108686, the entirety of which is incorporated herein by reference.

It can be difficult to remove C18 fatty acids, in particular alpha-linolenic acid (ALA) and/or gamma-linolenic acid (GLA), from feed mixtures efficiently without using large volumes of aqueous alcohol solvents. Efficient removal of C18 fatty acids is advantageous since many specifications for pharmaceutical and dietary oils require a low content of these fatty acids. For example, certain oil specifications for use in Japan require an ALA content of less than 1 wt %. Accordingly, this “mixed solvents” process is particularly useful as it can be employed to efficiently recover a PUFA product from a feed mixture whilst minimising the amount of C18 fatty acids, for example ALA and/or GLA, present in the resultant product. A C18 fatty acid is a C18 aliphatic monocarboxylic acid having a straight or branched hydrocarbon chain. Typical C18 fatty acids include stearic acid (C18:0), oleic acid (C18:1n9), vaccenic acid (C18: 1n7), linoleic acid (C18:2n6), gamma-linolenic acid/GLA (C18:3n6), alpha-linolenic acid/ALA (C18:3n3) and stearidonic acid/SDA (C18:4n3).

For the avoidance of doubt, typically in this “mixed solvents” process, the first chromatographic separation step is carried out in a chromatography apparatus as described herein, i.e. a chromatography apparatus comprising as the solid adsorbent phase a C18-bonded silica as described herein. Typically, in this “mixed solvents” process, the second chromatographic separation step is carried out in a chromatography apparatus as described herein, i.e. a chromatography apparatus comprising as the solid adsorbent phase a C18-bonded silica as described herein.

Preferably, in this “mixed solvents” process, both the first and second chromatographic separation steps are carried out in a chromatography apparatus as described herein, i.e. a chromatography apparatus operating at a pressure of less than 20 bar and comprising as the solid adsorbent phase a C18-bonded silica as described herein. However in an alternative embodiment of this “mixed solvents” process, only one of the first and/or second chromatographic separation steps is carried out in a chromatography apparatus as described herein, and the other separation step is carried out in a chromatography apparatus having a different solid adsorbent phase and/or operating pressure.

Thus, in this embodiment of the “mixed solvents” process, typically the first chromatographic separation step is carried out in a chromatography apparatus as described herein, i.e. a chromatography apparatus operating at a pressure of less than 20 bar and comprising as the solid adsorbent phase a C18-bonded silica as described herein, and the second chromatographic step is carried out in a chromatography apparatus having a different solid adsorbent phase, e.g. an alternative silica, such as an alternative C18-bonded silica, a C8-bonded silica, pure silica, cyano-bonded silica and phenyl-bonded silica, or a non-silica based solid adsorbent phase. Alternatively, the first chromatographic separation step is carried out in a chromatography apparatus as described herein and the second chromatographic step is carried out in a chromatography apparatus operating at a pressure of greater than 20 bar. Alternatively, the first chromatographic separation step is carried out in a chromatography apparatus as described herein and the second chromatographic step is carried out in a chromatography apparatus operating at a pressure of greater than 20 bar having a different solid adsorbent phase, e.g. an alternative silica, such as an alternative C18-bonded silica, a C8-bonded silica, pure silica, cyano-bonded silica and phenyl-bonded silica, or a non-silica based solid adsorbent phase.

Alternatively, in this embodiment of the “mixed solvents” process, the second chromatographic separation step may be carried out in a chromatography apparatus as described herein, i.e. a chromatography apparatus operating at a pressure of less than 20 bar and comprising as the solid adsorbent phase a C18-bonded silica as described herein, and the first chromatographic step is carried out in a chromatography apparatus having a different solid adsorbent phase, e.g. an alternative silica, such as an alternative C18-bonded silica, a C8-bonded silica, pure silica, cyano-bonded silica and phenyl-bonded silica, or a non-silica based solid adsorbent phase. Alternatively, the second chromatographic separation step is carried out in a chromatography apparatus as described herein and the first chromatographic step is carried out in a chromatography apparatus operating at a pressure of greater than 20 bar. Alternatively, the second chromatographic separation step is carried out in a chromatography apparatus as described herein and the first chromatographic step is carried out in a chromatography apparatus operating at a pressure of greater than 20 bar having a different solid adsorbent phase, e.g. an alternative silica, such as an alternative C18-bonded silica, a C8-bonded silica, pure silica, cyano-bonded silica and phenyl-bonded silica, or a non-silica based solid adsorbent phase.

Typically in this “mixed solvents” process, the first and second chromatographic separation steps are carried out in different chromatography apparatuses. Alternatively, the first and second chromatographic separation steps are carried out in the same chromatography apparatus.

Typically in this “mixed solvents” process, the second organic solvent has a polarity index which differs from the polarity index of the first organic solvent by 2.0 or less. Thus, where the polarity index of the first organic solvent is P1, the polarity index of the second organic solvent is P2, |P1−P2| is up to 2.0. The polarity index (P′) of a solvent is a well-known measure of how polar a solvent is. A higher polarity index figure indicates a more polar solvent. Polarity index is typically determined by measuring the ability of a solvent to interact with various test solutes. More typically, the polarity index (P′) of a solvent is as defined in Burdick and Jackson's Solvent Guide (AlliedSignal, 1997), the entirety of which is incorporated herein by reference. Burdick and Jackson rank solvents by reference to a numerical index that ranks solvents according to their different polarity. The Burdick and Jackson index is based on the structure of the solvents.

Preferably in this “mixed solvents” process, the second organic solvent has a polarity index which differs from the polarity index of the first organic solvent by between 0.1 and 2.0. For example, the second organic solvent may have a polarity index which differs from the polarity index of the first organic solvent by at least 0.2, at least 0.3, at least 0.4, at least 0.5, or at least 0.6. For example, the second organic solvent may have a polarity index which differs from the polarity index of the first organic solvent by at most 1.8, at most 1.5, at most 1.3, at most 1.0, or at most 0.8. For example, the second organic solvent may have a polarity index which differs from the polarity index of the first organic solvent by between 0.2 and 1.8, between 0.3 and 1.5, between 0.4 and 1.3, between 0.5 and 1.0, or between 0.6 and 0.8.

In this “mixed solvents” process, the first and second organic solvents may be any of the organic solvents disclosed herein. Typically, though, the first and second organic solvents are miscible with water. More typically, the first and second organic solvents have a polarity index of 3.9 or greater. Preferably, the first and second organic solvents are chosen from tetrahydrofuran, isopropyl alcohol, n-propyl alcohol, methanol, ethanol, acetonitrile, 1,4-dioxane, N,N-dimethyl formamide, and dimethylsulfoxide.

Typically in this “mixed solvents” process, the first organic solvent:water ratio is from 99.9:0.1 to 75:25 parts by volume, preferably from 99.5:0.5 to 80:20 parts by volume. If the first organic solvent is methanol, the methanol:water ratio is typically from 99.9:0.1 to 85:15 parts by volume, preferably from 99.5:0.5 to 88:12 parts by volume. If the first organic solvent is acetonitrile, the acetonitrile:water ratio is typically from 99:1 to 75:25 parts by volume, preferably from 96:4 to 80:20 parts by volume.

Typically in this “mixed solvents” process, the second organic solvent:water ratio is from 99.9:0.1 to 75:25 parts by volume, preferably from 93:7 to 85:15 parts by volume. If the second organic solvent is methanol, the methanol:water ratio is typically from 95:5 to 85:15 parts by volume, preferably from 93:7 to 90:10 parts by volume. If the second organic solvent is acetonitrile, the acetonitrile:water ratio is typically from 90:10 to 80:20 parts by volume, preferably from 88:12 to 85:15 parts by volume.

Typically in this “mixed solvents” process, one of the first and second organic solvents is acetonitrile.

Typically in this “mixed solvents” process, one of the first and second organic solvents is methanol.

Preferably in this “mixed solvents” process, the first and second organic solvents are selected from acetonitrile and methanol. Thus, it is preferable that (i) the first organic solvent is methanol and the second organic solvent is acetonitrile, or (ii) the first organic solvent is acetonitrile and the second organic solvent is methanol.

More preferably in this “mixed solvents” process, the first organic solvent is methanol and the second organic solvent is acetonitrile, and (a) the methanol:water ratio is from 99.9:0.1 to 85:15 parts by volume, preferably from 99.5:0.5 to 88:12 and/or (b) the acetonitrile:water ratio is from 90:10 to 80:20 parts by volume, preferably from 88:12 to 85:15 parts by volume. In certain embodiments it is preferable that (a) the methanol:water ratio is from 91:9 to 93:7 parts by volume, and/or (b) the acetonitrile:water ratio is from 86:14 to 88:12 parts by volume.

Alternatively in this “mixed solvents” process, the first organic solvent is acetonitrile and the second organic solvent is methanol, and (a) the acetonitrile:water ratio is from 99:1 to 75:25 parts by volume, preferably 96:4 to 80:20 parts by volume, and/or (b) the methanol:water ratio is from 95:5 to 85:15 parts by volume, preferably from 93:7 to 90:10 parts by volume. In certain embodiments it is preferable that (a) the acetonitrile:water ratio is from 86:14 to 88:12 parts by volume, and/or (b) the methanol:water ratio is from 87:13 to 89:11 parts by volume.

Typically in this “mixed solvents” process, the first organic solvent is acetonitrile, and the intermediate product has a lower concentration of one or more of the C18 fatty acid impurities disclosed above than the feed mixture. Alternatively in this “mixed solvents” process, the second organic solvent is acetonitrile, and the PUFA product produced in the second separation step has a lower concentration of one or more of the C18 fatty acid impurities disclosed above than the intermediate product.

Preferably in this “mixed solvents” process, the PUFA product is EPA ethyl ester, and (i) the first organic solvent is acetonitrile, and the intermediate product has a lower concentration of one or more of the C18 fatty acid impurities disclosed above than the feed mixture, or (ii) the second organic solvent is acetonitrile, and the PUFA product produced in the second separation step has a lower concentration of one or more of the C18 fatty acid impurities disclosed above than the intermediate product.

More preferably in this “mixed solvents” process, the PUFA product is EPA ethyl ester, and (i) the first organic solvent is acetonitrile, the second organic solvent is methanol and the intermediate product has a lower concentration of one or more of the C18 fatty acid impurities disclosed above than the feed mixture, or (ii) the first organic solvent is methanol, the second organic solvent is acetonitrile, and the PUFA product produced in the second separation step has a lower concentration of one or more of the C18 fatty acid impurities disclosed above than the intermediate product.

Any known chromatography apparatus can be employed in this “mixed solvents” process. Typically the first and/or second separation steps are carried out using either a stationary bed chromatography apparatus or one or more simulated or actual moving bed chromatography apparatuses as described herein.

Typically in this “mixed solvents” process, the first chromatographic separation step comprises introducing the feed mixture into a stationary bed chromatography apparatus and the second chromatographic separation step comprises introducing the intermediate product into a stationary bed chromatography apparatus. Thus, typically in this “mixed solvents” process, the first chromatographic separation step is carried out using a stationary bed chromatography apparatus and the second chromatographic separation step is carried out using a stationary bed chromatography apparatus.

Alternatively in this “mixed solvents” process, the first chromatographic separation step comprises introducing the feed mixture into a stationary bed apparatus and the second chromatographic separation step comprises introducing the intermediate product into a simulated or actual moving bed chromatography apparatus. Thus, typically in this “mixed solvents” process, the first chromatographic separation step is carried out using a stationary bed apparatus and the second chromatographic separation step is carried out using a simulated or actual moving bed chromatography apparatus.

Alternatively in this “mixed solvents” process, the first chromatographic separation step comprises introducing the feed mixture into a simulated or actual moving bed chromatography apparatus and the second chromatographic separation step comprises introducing the intermediate product into a stationary bed chromatography apparatus. Thus, typically in this “mixed solvents” process, the first chromatographic separation step is carried out using a simulated or actual moving bed chromatography apparatus and the second chromatographic separation step is carried out using a stationary bed chromatography apparatus.

Alternatively in this “mixed solvents” process, the first chromatographic separation step comprises introducing the feed mixture into a simulated or actual moving bed chromatography apparatus and the second chromatographic separation step comprises introducing the intermediate product into a simulated or actual moving bed chromatography apparatus. Thus, typically in this “mixed solvents” process, the first chromatographic separation step is carried out using a simulated or actual moving bed chromatography apparatus and the second chromatographic separation step is carried out using a simulated or actual moving bed chromatography apparatus.

In this “mixed solvents” process, said first chromatographic separation step may consist of a single chromatographic separation or two or more chromatographic separations, provided that each separation uses as eluent a mixture of water and the first organic solvent.

In this “mixed solvents” process, said second chromatographic separation step may consist of a single chromatographic separation or two or more chromatographic separations, provided that each separation uses as eluent a mixture of water and the second organic solvent.

In this “mixed solvents” process, the first and second separation steps may be carried out at the same temperature or a different temperature, preferably the same temperature.

Typically in this “mixed solvents” process, each chromatographic separation step involves passing a feed mixture through one or more chromatographic columns, and the temperature of at least one of those chromatographic columns is greater than room temperature. More typically, the temperature of all of the chromatographic columns used is greater than room temperature. Preferred temperatures are as described above.

In this “mixed solvents” process, when the first and/or second chromatographic separation step is carried out in a simulated moving bed apparatus, at least one of the first and/or second chromatographic separation steps typically involves at least one, for example one, “single pass” SMB step as described above.

In this “mixed solvents” process, when the first and/or second chromatographic separation step is carried out in a simulated moving bed apparatus, at least one of the first and/or second chromatographic separation steps typically involves at least one, for example one, “double pass” SMB step as described above.

In the “mixed solvents” process, reference to an “input stream” above in the context of the “double pass” mode of operating an SMB separation refers to the feed mixture when the above-described SMB process is used in the first chromatographic separation step, and refers to the intermediate product when the above-described SMB process is used in the second chromatographic separation step.

In the “mixed solvents” process, reference to an “aqueous organic solvent” above in the context of the “double pass” mode of operating an SMB separation refers to the mixture of water and the first organic solvent when the above-described SMB process is used in the first chromatographic separation step, and refers to the mixture of water and the second organic solvent when the above-described SMB process is used in the second chromatographic separation step.

In this “mixed solvents” process, when the first and/or second chromatographic separation step is carried out in a simulated moving bed apparatus, at least one of the first and/or second chromatographic separation steps typically involves a “back-to-back” SMB process as described above.

For the avoidance of doubt, in the “mixed solvents” process, if the first chromatographic separation step is a “back-to-back” SMB process along the above lines, the eluent in each of the SMB steps is a mixture of water and the first organic solvent. If the second chromatographic separation step is a “back-to-back” SMB process along the above lines, the eluent in each of the SMB steps is a mixture of water and the second organic solvent.

In the “mixed solvents” process, reference to an “input stream” above in the context of the “back-to-back” mode of operating an SMB separation refers to the feed mixture when the above-described SMB process is used in the first chromatographic separation step, and refers to the intermediate product when the above-described SMB process is used in the second chromatographic separation step.

In the “mixed solvents” process, reference to an “aqueous organic solvent” above in the context of the “back-to-back” mode of operating an SMB separation refers to the mixture of water and the first organic solvent when the above-described “back-to-back” SMB process is used in the first chromatographic separation step, and refers to the mixture of water and the second organic solvent when the above-described “back-to-back” SMB process is used in the second chromatographic separation step. The organic solvent used in the first and second SMB steps is the same. The organic solvent:water ratio used in the first and second SMB steps may be the same or different.

In the “mixed solvents” process, reference to a “second product” above in the context of the “back-to-back” mode of operating an SMB separation refers to the intermediate product when the above-described SMB process is used in the first chromatographic separation step, and refers to the PUFA product when the above-described SMB process is used in the second chromatographic separation step.

Preferably in the “mixed solvents” process, the PUFA product is EPA ethyl ester, and: (i) the first organic solvent is acetonitrile, the second organic solvent is methanol, and the first chromatographic separation step comprises introducing the feed mixture into a stationary bed apparatus and the second chromatographic separation step comprises introducing the intermediate product into a simulated or actual moving bed chromatography apparatus; or (ii) the first organic solvent is methanol and the second organic solvent is acetonitrile, the first chromatographic separation step comprises introducing the feed mixture into a simulated or actual moving bed chromatography apparatus and the second chromatographic separation step comprises introducing the intermediate product into a stationary bed chromatography apparatus.

Even more preferably in this “mixed solvents” process, the PUFA product is EPA ethyl ester, and:
(i) the first organic solvent is acetonitrile, the second organic solvent is methanol, the intermediate product has a lower concentration of one or more of the C18 fatty acid impurities disclosed above than the feed mixture, and the first chromatographic separation step comprises introducing the feed mixture into a stationary bed apparatus and the second chromatographic separation step comprises introducing the intermediate product into a simulated or actual moving bed chromatography apparatus; or
(ii) the first organic solvent is methanol, the second organic solvent is acetonitrile, the PUFA product produced in the second separation step has a lower concentration of one or more of the C18 fatty acid impurities disclosed above than the intermediate product, and the first chromatographic separation step comprises introducing the feed mixture into a simulated or actual moving bed chromatography apparatus and the second chromatographic separation step comprises introducing the intermediate product into a stationary bed chromatography apparatus.

The following examples illustrate the invention.

### EXAMPLES

### Example 1: Particle Characterisation of Different Silicas

Four different silicas were selected for testing. The physical parameters of each of these silicas were first measured as described below. The results are collated in Table 1. All silicas are commercially available and were obtained from the respective supplier. Silica 1 is selected as a reference example, and the other three silicas are examples of silicas for use according to the present invention.

TABLE 1
Comparison of the physical properties of
five different silica specifications

| | 1 (reference | | | |
|---|---|---|---|---|
| Silica | example) | 2 | 3 | 4 |
| Carbon loading (%) | 24.9 | 19.8 | 20.1 | 17.8 |
| Surface area | 542 | 417 | 519 | 339 |
| (m²/g) | | | | |
| Dv(10) (μm) | 150 | 150 | 168 | 187 |
| D[4.3] (μm) | 228 | 248 | 251 | 257 |
| Dv(90) (μm) | 320 | 365 | 347 | 339 |
| Bulk density | 0.713 | 0.543 | 0.671 | 0.654 |
| (kg/dm³) | | | | |

The % carbon loading was determined by combustion analysis. The surface area is a measure of the surface area of the unbonded silica (i.e. not bound to octadecyl carbon chains). It was determined in porosity experiments by BET surface area analysis.

Dv(10), D[4,3] and Dv(90) were determined by laser diffraction. The measurements were taken in aqueous suspension using a Malvern Mastersizer 2000 instrument.

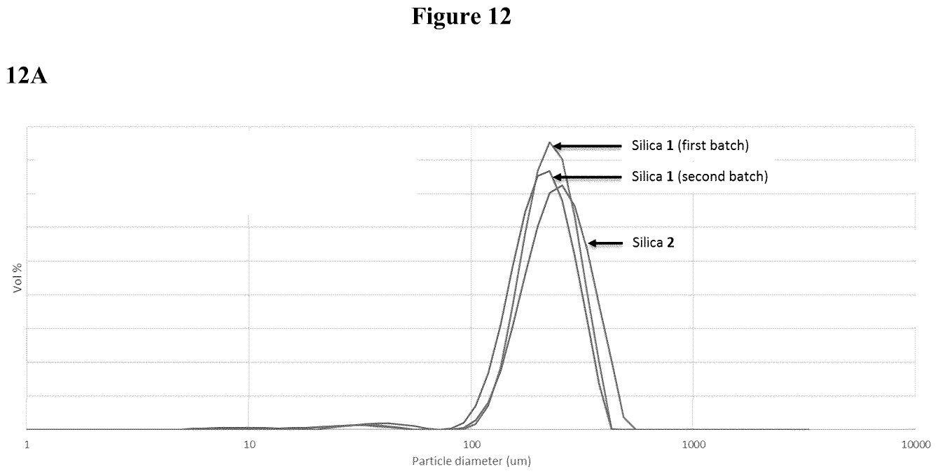

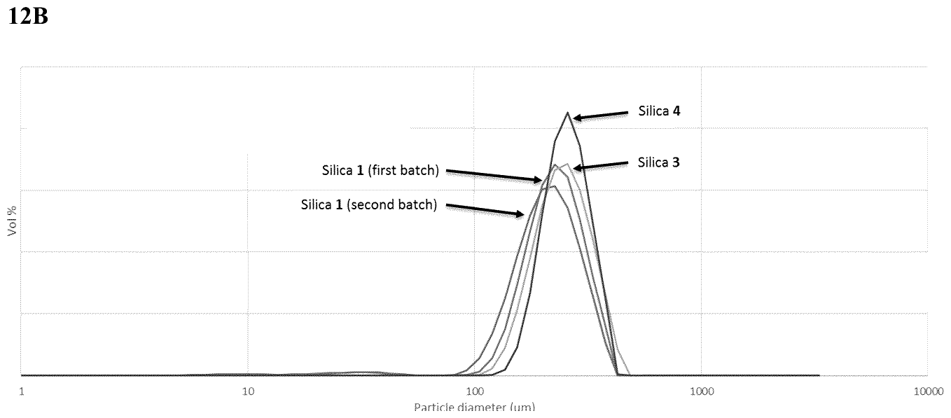

Plots showing the total contribution to particle volume of a silica sample as a function of particle diameter for different C18 silica types are shown in FIG. 12. These are measures of the particle distribution of each sample.

FIG. 12A compares the particle distribution of silica 1 (2 separate batches) and silica 2. It can be observed that both of these silicas contain a significant number of smaller particles, with a diameter of from 10 to 100 μm (note that although the peak corresponding to these particles is small, the y-axis reflects the total volume contribution to the overall sample of these particles and not the total number of particles of this diameter present; as these particles are small their contribution to the overall volume is small, but their number is significant). Thus, these silicas have a broad particle distribution with a significant “tail” of smaller particles.

FIG. 12B compares the particle distribution of silica 1 (2 separate batches), silica 3 and silica 4. It can be observed that silica 3 and silica 4 both contain a negligible number of small particles with a diameter of from 10 to 100 μm, in contrast to silica 1. This is reflected in the higher Dv(10) value reported for silica 3 and silica 4 in Table 1 compared with silica 1. It can also be seen that overall, silica 3 and silica 4 have a significantly narrower particle size distribution than silica 1.

The bulk particle density was determined by recording the weight of material packed into a column with a defined volume.

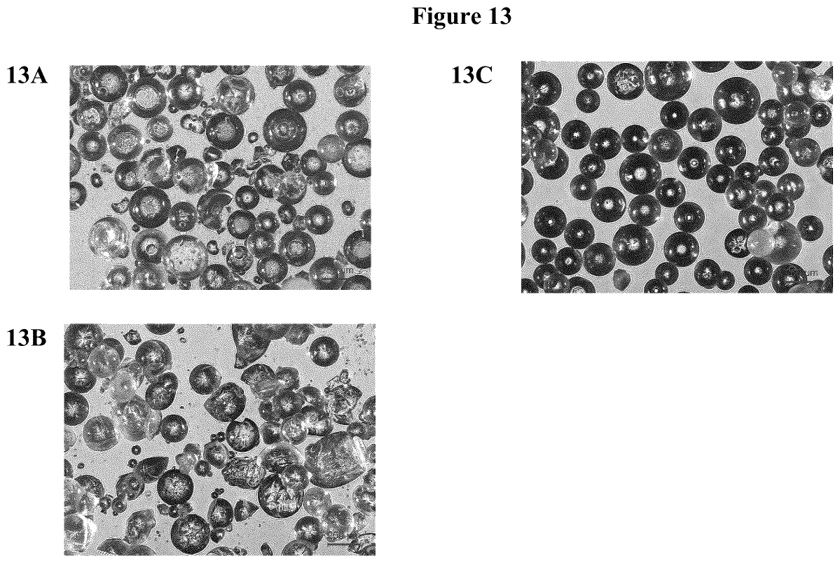

Light microscope images of silicas 1, 2 and 3 were also obtained, and are shown in FIG. 13. As can be seen from FIGS. 13A and 13B, silicas 1 and 2 contain broken particles, with a number of particles having a small diameter present. In contrast, silica 3 (FIG. 13C) shows no evidence of broken particles, and has a more uniform particle size distribution.

### Example 2: Comparison of Peak Resolution and Pressure Drops Using Different Silicas

A fish-oil derived feedstock (comprising about 74 GC-area % EPA and about 10 GC-area % DHA) was pulse injected on a single stationary bed chromatography apparatus composed of 3 columns in series, each with a diameter of 10 mm and a length of 250 mm. Each of the silicas set out in Table 2 was employed in turn as the stationary phase and a mixture of methanol:water (90:10) was employed as eluent.

The operating parameters and flowrates were as follows:
Feed mixture pulse: 0.5 mL of 25 wt.-% of fish oil derived feedstock dissolved in methanol Eluent feed rate (D1): 7 mL/min methanol:water at 90:10 vol. %

In each of the column pulse experiments, the following parameters were measured:
The pressure drop along the column (using the experiment with silica 1 as reference);
The retention time of EPA versus the reference experiment;
The selectivity (i.e. difference in retention times) for the αSDA/EPA peaks;
The selectivity for the αDHA/EPA peaks;
The asymmetry of the EPA peak; and
The height of equivalent theoretical plates (HETP) for the EPA peak.

The results are shown in Table 2 (each of the silicas used is as per the specification in Table 1; for brevity, the shortened reference name for each silica has been used).

TABLE 2
Comparison of the performance of each of the five silica samples
from pulse experiment with an EPA-containing feed mixture

| | 1 (reference | | | |
|---|---|---|---|---|
| Silica | example) | 2 | 3 | 4 |
| Pressure drop vs. | 100% | 88% | 115% | 70% |
| reference | | | | |
| Selectivity αSDA/EPA | 0.76 | 0.75 | 0.75 | 0.75 |
| Selectivity αDHA/EPA | 1.34 | 1.35 | 1.38 | 1.32 |
| Asymmetry in EPA | 1.25 | 1.18 | 1.08 | 1.04 |
| peak | | | | |
| HETP of EPA peak | 3.28 | 1.96 | 1.86 | 1.32 |
| (cm) | | | | |

All of the tested silicas demonstrated an acceptable pressure drop over the length of the chromatography apparatus compared to the reference example. Silicas 2 and 4 result in a lower pressure drop over the length of the apparatus than silica 1, with silica 4 being particularly advantageous in this regard.

All of the tested silicas demonstrated an acceptable selectivity of the EPA peak over the DHA and SDA peaks. The selectivity was calculated as the ratio of the retention times of the relevant pair of peaks.

The peak asymmetry (As) of the EPA peak was determined by dividing the peak width after the peak centre by the peak width before the peak centre at 10% peak height. All of the tested silicas showed an improved symmetry of the EPA peak compared to the reference example (silica 1). Thus, each of these silicas provides an improved peak shape of the EPA, because a more symmetrical peak means that the EPA peak does not “tail” so much into other peaks.

This allows for a more efficient separation, because less eluent (i.e. a lower eluent dilution) is required to obtain a high yield of a desired product having a specific purity.

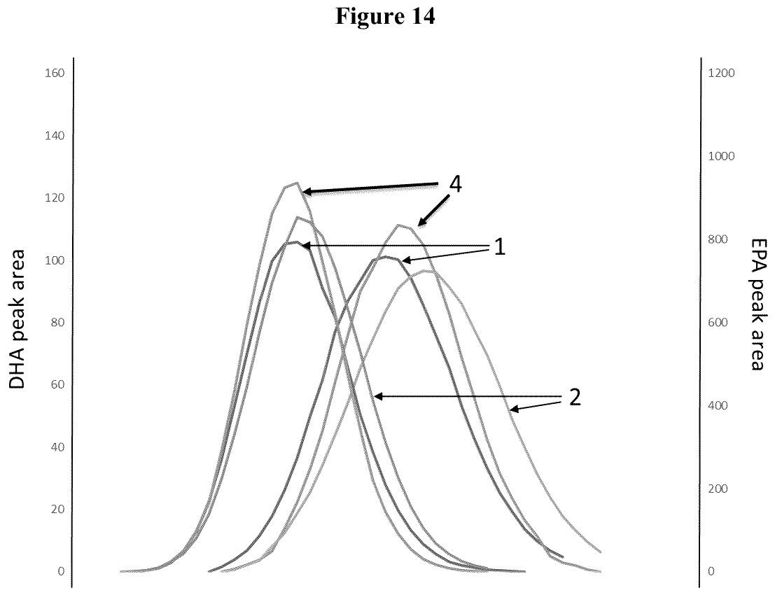

A graphical representation of the asymmetry of the EPA peak for each of the silicas 1, 2 and 4, from which the values in Table 2 were calculated, is shown in FIG. 14.

The HETP values were calculated using the following equations:

$$N = 5.545 {( \frac{{t}_{R}}{{ω}_{h}} )}^{2} and HETP = \frac{L}{N}$$

wherein:
   N=number of theoretical plates
   T_{R}=retention time
   ω_{R}=peak width at half height (in units of time)
L=length of column (mm)

The number of theoretical plates is a mathematical concept as chromatography columns do not contain anything resembling physical distillation plates or other similar features. Columns with high plate numbers are considered to be more efficient; a column with a high number of theoretical plates will have a narrower peak at a given retention time than a column with a lower N number (i.e. a lower HETP). A low HETP value is therefore indicative of well-resolved peaks that leads to higher column efficiency, meaning that less eluent is required to obtain a high yield of a desired product having a specific purity.

All test silicas 2 to 4 show an improved HETP compared to the reference silica 1.

The following conclusions can be drawn from the data in Tables 1 and 2:
Silicas with a more uniform particle size distribution, and in particular those with a low number of particularly small particles (reflected in Dv(10) values), display an improved resolution of the PUFA peaks. This can be seen from a comparison of the HETP values of silicas 2 and 3, which have very similar carbon loading.
Silicas with a lower carbon loading display an improved resolution of the PUFA peaks. This can be seen from a comparison of the HETP values of silicas 1 and 2, both of which have similar particle distributions with a “tail” of small particles, yet nonetheless the HETP value for silica 2 is significantly lower than that for silica 1. Likewise, silicas 3 and 4 have similar particle size distributions, but silica 4 has a lower carbon loading and an improved HETP value.
   Silicas with a lower surface area display an improved resolution of the PUFA peaks. In general, a positive correlation is observed between carbon loading and total surface area.

### Example 3: Comparison of Low-Pressure EPA Separation Via SMB Using Silica 4 with HPLC/Batch Processes

Three purifications of an EPA-containing feedstock were effected using silica 4 in an SMB process operating at reduced pressure with 8 columns, each having diameter 1.6 cm and length 25 cm, and compared with batch/HPLC separation using various silicas that are reported in the literature. The productivity and eluent dilution of each process was measured. The results are described in Table 3.

Each of the three purifications was carried out using either a single pass SMB or a double pass SMB with both a “fast” cut (pass one) and a “slow” cut (pass two), to remove the faster and slower running components than EPA, respectively. The operating parameters for each purification were as follows:

### Test run A

Feedstock: 92 GC-area % EPA ethyl ester content
Desired product: 97 GC-area % EPA ethyl ester
Process: Single pass SMB
Eluent: Methanol:water (91:9)
Temperature: 40° C.
Pressure: <10 bar
Feed mixture feed rate (F): 0.5 mL/min of 50 wt.-% fish oil feedstock in methanol
Eluent feed rate (D): 17.3 mL/min
Extract rate: 13.2 mL/min
Raffinate rate: 4.6 mL/min

### Test run B

Feedstock: 74 GC-area % EPA ethyl ester content
Desired product: 97 GC-area % EPA ethyl ester
Process: Double pass SMB
Eluent (slow cut): Methanol:water (91:9)
Eluent (fast cut): Methanol:water (90:10)
Temperature: 40° C.
Pressure: <10 bar

### Pass One

Feed mixture feed rate (F1): 0.5 mL/min of 50 wt. % fish oil feedstock in methanol
Eluent feed rate (D1): 22.0 mL/min
Extract rate (E1): 14.8 mL/min
Raffinate rate (R1): 7.7 mL/min

### Pass Two

Feed mixture feed rate (F2): 1.0 mL/min of 50 wt. % of raffinate from slow cut in methanol
Eluent feed rate (D2): 13.5 mL/min
Extract rate (E2): 12.3 mL/min
Raffinate rate (R2): 2.3 mL/min

### Test Run C

Feedstock: 74 GC-area % EPA ethyl ester content
Desired product: 97 GC-area % EPA ethyl ester
Process: Double pass SMB
Eluent (slow cut): Methanol:water (90:10)
Eluent (fast cut): Methanol:water (91:9)
Temperature: 40° C.
Pressure: <6 bar

### Pass One

Feed mixture feed rate (F1): 0.35 mL/min of 50 wt. % fish oil feedstock in methanol
Eluent feed rate (D1): 13.9 mL/min
Extract accumulation rate (E1): 10.1 mL/min
Raffinate accumulation rate (R1): 4.2 mL/min

### Pass Two

Feed mixture feed rate (F2): 0.7 mL/min of 50 wt. % of raffinate from slow cut in methanol
Eluent feed rate (D2): 8.9 mL/min
Extract rate (E2): 8.5 mL/min
Raffinate rate (R2): 1.1 mE/min

As can be seen from Table 3, the chromatographic separation processes of the present invention lead to a significantly reduced dilution of solvent per kg EPA product, i.e. a much more efficient separation requiring less solvent, than all the comparative prior art processes which utilise higher operating pressures. At the same time, a reasonable level of productivity is maintained, which is surprising, because it is generally known in the art that at reduced operating pressures up to a tenfold reduction in chromatographic separation productivity is ordinarily observed.

It is also notable that such a low level of dilution and high yield of EPA product can be obtained using such a silica having such a large average particle size. It is generally known in the art that larger particle size leads to a larger HETP and loss in peak resolution. Thus, the discovery of the present inventors that certain types of silicas having larger average particle sizes can be used and still achieve a high yield and acceptable productivity in PUFA production, and moreover provide a lower dilution of solvent, is surprising.

TABLE 3
Operating parameters and output productivity, dilution and yields for chromatographic separations
of EPA according to the present invention and in comparative prior art separations

| | | | | | Comp. 1 | Comp. 2 | Comp. 3 |
|---|---|---|---|---|---|---|---|
| Example | A | B | C | D (reference) | Novasep Cyclojet | Novasep Varicol | Nissui |
| Prior art reference | — | — | — | — | U.S. Pat. No. | U.S. Pat. No. | U.S. Pat. No. |
| | | | | | 9,802,882 Ex. | 9,802,882 Ex. | 5,840,944 |
| | | | | | 1a/2a | 1b/2b | |
| Type of separation | SMB | SMB | SMB | SMB | Batch/HPLC | Varicol | Batch |
| | | | | | | (SMB) | |
| Silica | 4 | 4 | 4 | 1 | C18 particle size | C18 particle size | C18 particle |
| | | | | | 20 μm | 20 μm | size 10-20 μm |
| Pressure | <10 bar | <10 bar | <6 bar | | HPLC | HPLC | HPLC |

Pass One

| | | | | | | | |
|---|---|---|---|---|---|---|---|
| Solvent | MeOH:H₂O | MeOH:H₂O | MeOH:H₂O | MeOH:H₂O | MeOH:H₂O | MeOH:H₂O | EtOH |
| Productivity | 0.93 | 0.80 | 0.55 | 0.26 | 2.76 | 2.46 | 0.38 |
| (kg_{EPA}/kg_{ads}/day) | | | | | | | |
| Dilution | 97 | 141 | 131 | 197 | 224 | 258 | 1204 |
| (Lₛₒₗᵥₑₙₜ/kg_{EPA}) | | | | | | | |
| EPA recovery (yield) | >97% | >98% | >98% | 92% | 95% | 95% | 85% |

Pass Two

| | | | | | | | |
|---|---|---|---|---|---|---|---|
| Solvent | — | MeOH:H₂O | MeOH:H₂O | MeOH:H₂O | Acetone:H₂O | Acetone:H₂O | — |
| Productivity | — | 2.04 | 1.47 | 0.39 | 3.58 | 3.35 | — |
| (kg_{EPA}/kg_{ads}/day) | | | | | | | |
| Dilution (Lₛₒₗᵥₑₙₜ/kg_{EPA}) | — | 35.2 | 32.0 | 96 | 153 | 0 | — |
| EPA recovery (yield) | — | >99% | >99% | 98% | 95% | 95% | — |
| Productivity | 0.93 | 0.58 | 0.40 | 0.16 | 1.56 | 1.42 | 0.38 |
| (kg_{EPA}/kg_{ads}/day) | | | | | | | |
| Dilution (Lₛₒₗᵥₑₙₜ/kg_{EPA}) | 97 | 176 | 163 | 293 | 377 | 258 | 1204 |
| EPA recovery (yield) | >97.0% | >97.0% | >97.0% | 90% | 90.3% | 90.3% | 85.0% |

1.-17. (canceled)

18. A chromatographic separation process for recovering a polyunsaturated fatty acid (PUFA) product from a feed mixture, which comprises introducing the feed mixture into a chromatography apparatus comprising one or more chromatographic columns containing:
(a) a liquid eluent phase which is an aqueous organic solvent; and
(b) a solid adsorbent phase which is a C18-bonded silica,
wherein the pressure within the one or more chromatographic columns is less than 20 bar, thereby resulting in purification of the feed mixture,
further wherein:
(1) the silica has an average particle diameter of from 230 to 270 μm and a Dv(10) of 160 μm or greater; and/or
(2) the silica has a carbon loading of from 15 to 24 wt %; and/or
(3) the silica has a surface area of 500 m²/g or less.

19. A chromatographic separation process according to claim 18, wherein the silica has a carbon loading of from 15 to 24 wt %, and:
the silica has an average particle diameter of from 230 to 270 μm and a Dv(10) of 160 μm or greater; and/or
the silica has a surface area of 500 m²/g or less.

20. A chromatographic separation process according to claim 18, wherein the silica has an average particle diameter of from 230 to 270 μm.

21. A chromatographic separation process according to claim 18, wherein the silica has a Dv(10) of 160 μm or greater and/or a Dv(10) of 225 μm or less.

22. A chromatographic separation process according to claim 18, wherein the silica has an average particle diameter of from 230 to 270 μm and a Dv(10) of from 160 to 225 μm.

23. A chromatographic separation process according to claim 18, wherein the silica has a carbon loading of from 15 to 24 wt %.

24. A chromatographic separation process according to claim 18, wherein the silica has a carbon loading of from 16 to 22 wt %.

25. A chromatographic separation process according to claim 18, wherein the silica has a surface area of 500 m²/g or less and/or a surface area of 200 m²/g or more.

26. A chromatographic separation process according to claim 18, wherein the silica:
(a) has a Dv(10) of 170 μm or greater; and/or
(b) has an average particle diameter of from 250 to 260 μm; and/or
(c) has a carbon loading of from 17 to 18.5 wt %; and/or
(d) has a pore diameter of from 80 to 140 Å; and/or
(e) has a bulk density of 0.7 kg/dm³ or less.

27. A chromatographic separation process according to claim 18, wherein at least 80% of the silica particles by volume have a diameter of from 200 to 500 μm.

28. A chromatographic separation process according to claim 18, wherein the aqueous organic solvent is a mixture of water and an alcohol, an ether, an ester, a ketone or a nitrile.

29. A chromatographic separation process according to claim 18, wherein the pressure within the one or more chromatographic columns is less than 10 bar.

30. A chromatographic separation process according to claim 18, wherein the chromatography apparatus is an actual or simulated moving bed (SMB) chromatography apparatus.

31. A chromatographic separation process according to claim 18, wherein the feed mixture is (i) a natural or synthetic feedstock comprising at least one PUFA product, or a feedstock which is derived from microorganisms or fungi, or (ii) a partially purified feedstock comprising at least one PUFA product obtained from partial purification of a natural or synthetic feedstock, wherein said partially purified feedstock is optionally obtained by crystallisation, molecular distillation or fractional distillation, urea fractionation, extraction with silver nitrate or other metal salt solutions, iodolactonisation, supercritical fluid fractionation, or chromatography.

32. A chromatographic separation process according to claim 18, wherein:
(a) introducing the feed mixture into the chromatography apparatus results in purification of the feed mixture to yield the PUFA product; or
(b) introducing the feed mixture into the chromatography apparatus results in purification of the feed mixture to yield an intermediate product, wherein said intermediate product is subjected to further purification to obtain the PUFA product.

33. A chromatographic separation process according to claim 18, wherein the PUFA product comprises a PUFA or a PUFA derivative, wherein the derivative is a mono-, di- or tri-glyceride, ester, phospholipid, amide, lactone or salt of the PUFA.

34. A chromatographic separation process according to claim 18, wherein:
(a) the yield of PUFA product obtained from the chromatographic separation process, based on the total mass of that PUFA product present in the feed mixture, is greater than 80 wt %; and/or
(b) the PUFA product is produced in a purity of greater than 80 wt %.
